SEQUENCE LISTING (23 sequences)

SEQ ID NO: 1 - protein, 596 aa, Arabidopsis thaliana
MFALSKVLRRTQRLRLGACSAVFSKDIQLGGERSFDSNSIASTKREAVPRFYEISSLSNRALSSSAGTKSDQEEDDLEDGFSELEGSKSGQGSTSSDEDEGKLSADEEEEEELDLIETDVSRKTVEKKQSELFKTIVSAPGLSIGSALDKWVEEGNEITRVEIAKAMLQLRRRRMYGRALQMSEWLEANKKIEMTERDYASRLDLTVKIRGLEKGEACMQKIPKSFKGEVLYRTLLANCVAAGNVKKSELVFNKMKDLGFPLSGFTCDQMLLLHKRIDRKKIADVLLLMEKENIKPSLLTYKILIDVKGATNDISGMEQILETMKDEGVELDFQTQALTARHYSGAGLKDKAEKVLKEMEGESLEANRRAFKDLLSIYASLGREDEVKRIWKICESKPYFEESLAAIQAFGKLNKVQEAEAIFEKIVKMDRRASSSTYSVLLRVYVDHKMLSKGKDLVKRMAESGCRIEATTWDALIKLYVEAGEVEKADSLLDKASKQSHTKLMMNSFMYIMDEYSKRGDVHNTEKIFLKMREAGYTSRLRQFQALMQAYINAKSPAYGMRDRLKADNIFPNKSMAAQLAQGDPFKKTAISDILD

SEQ ID NO: 2 - protein, 918 aa, Arabidopsis thaliana
MASTPPHWVTTTNNHRPWLPQRPRPGRSVTSAPPSSSASVSSAHLSQTTPNFSPLQTPKSDFSGRQSTRFVSPATNNHRQTRQNPNYNHRPYGASSSPRGSAPPPSSVATVAPAQLSQPPNFSPLQTPKSDLSSDFSGRRSTRFVSKMHFGRQKTTMATRHSSAAEDALQNAIDFSGDDEMFHSLMLSFESKLCGSDDCTYIIRELGNRNECDKAVGFYEFAVKRERRKNEQGKLASAMISTLGRYGKVTIAKRIFETAFAGGYGNTVYAFSALISAYGRSGLHEEAISVFNSMKEYGLRPNLVTYNAVIDACGKGGMEFKQVAKFFDEMQRNGVQPDRITFNSLLAVCSRGGLWEAARNLFDEMTNRRIEQDVFSYNTLLDAICKGGQMDLAFEILAQMPVKRIMPNVVSYSTVIDGFAKAGRFDEALNLFGEMRYLGIALDRVSYNTLLSIYTKVGRSEEALDILREMASVGIKKDVVTYNALLGGYGKQGKYDEVKKVFTEMKREHVLPNLLTYSTLIDGYSKGGLYKEAMEIFREFKSAGLRADVVLYSALIDALCKNGLVGSAVSLIDEMTKEGISPNVVTYNSIIDAFGRSATMDRSADYSNGGSLPFSSSALSALTETEGNRVIQLFGQLTTESNNRTTKDCEEGMQELSCILEVFRKMHQLEIKPNVVTFSAILNACSRCNSFEDASMLLEELRLFDNKVYGVVHGLLMGQRENVWLQAQSLFDKVNEMDGSTASAFYNALTDMLWHFGQKRGAELVALEGRSRQVWENVWSDSCLDLHLMSSGAARAMVHAWLLNIRSIVYEGHELPKVLSILTGWGKHSKVVGDGALRRAVEVLLRGMDAPFHLSKCNMGRFTSSGSVVATWLRESATLKLLILHDHITTATATTTTMKSTDQQQRKQTSFALQPLLL

SEQ ID NO: 3 - protein, 862 aa, Arabidopsis thaliana
MNLAIPNPNSHHLSFLIQNSSFIGNRRFADGNRLRFLSGGNRKPCSFSGKIKAKTKDLVLGNPSVSVEKGKYSYDVESLINKLSSLPPRGSIARCLDIFKNKLSLNDFALVFKEFAGRGDWQRSLRLFKYMQRQIWCKPNEHIYTIMISLLGREGLLDKCLEVFDEMPSQGVSRSVFSYTALINAYGRNGRYETSLELLDRMKNEKISPSILTYNTVINACARGGLDWEGLLGLFAEMRHEGIQPDIVTYNTLLSACAIRGLGDEAEMVFRTMNDGGIVPDLTTYSHLVETFGKLRRLEKVCDLLGEMASGGSLPDITSYNVLLEAYAKSGSIKEAMGVFHQMQAAGCTPNANTYSVLLNLFGQSGRYDDVRQLFLEMKSSNTDPDAATYNILIEVFGEGGYFKEVVTLFHDMVEENIEPDMETYEGIIFACGKGGLHEDARKILQYMTANDIVPSSKAYTGVIEAFGQAALYEEALVAFNTMHEVGSNPSIETFHSLLYSFARGGLVKESEAILSRLVDSGIPRNRDTFNAQIEAYKQGGKFEEAVKTYVDMEKSRCDPDERTLEAVLSVYSFARLVDECREQFEEMKASDILPSIMCYCMMLAVYGKTERWDDVNELLEEMLSNRVSNIHQVIGQMIKGDYDDDSNWQIVEYVLDKLNSEGCGLGIRFYNALLDALWWLGQKERAARVLNEATKRGLFPELFRKNKLVWSVDVHRMSEGGMYTALSVWLNDINDMLLKGDLPQLAVVVSVRGQLEKSSAARESPIAKAAFSFLQDHVSSSFSFTGWNGGRIMCQRSQLKQLLSTKEPTSEESENKNLVALANSPIFAAGTRASTSSDTNHSGNPTQRRTRTKKELAGSTA

SEQ ID NO: 4 - protein, 798 aa, Arabidopsis thaliana
MDASVVRFSQSPARVPPEFEPDMEKIKRRLLKYGVDPTPKILNNLRKKEIQKHNRRTKRETESEAEVYTEAQKQSMEEEARFQTLRREYKQFTRSISGKRGGDVGLMVGNPWEGIERVKLKELVSGVRREEVSAGELKKENLKELKKILEKDLRWVLDDDVDVEEFDLDKEFDPAKRWRNEGEAVRVLVDRLSGREINEKHWKFVRMMNQSGLQFTEDQMLKIVDRLGRKQSWKQASAVVHWVYSDKKRKHLRSRFVYTKLLSVLGFARRPQEALQIFNQMLGDRQLYPDMAAYHCIAVTLGQAGLLKELLKVIERMRQKPTKLTKNLRQKNWDPVLEPDLVVYNAILNACVPTLQWKAVSWVFVELRKNGLRPNGATYGLAMEVMLESGKFDRVHDFFRKMKSSGEAPKAITYKVLVRALWREGKIEEAVEAVRDMEQKGVIGTGSVYYELACCLCNNGRWCDAMLEVGRMKRLENCRPLEITFTGLIAASLNGGHVDDCMAIFQYMKDKCDPNIGTANMMLKVYGRNDMFSEAKELFEEIVSRKETHLVPNEYTYSFMLEASARSLQWEYFEHVYQTMVLSGYQMDQTKHASMLIEASRAGKWSLLEHAFDAVLEDGEIPHPLFFTELLCHATAKGDFQRAITLINTVALASFQISEEEWTDLFEEHQDWLTQDNLHKLSDHLIECDYVSEPTVSNLSKSLKSRCGSSSSSAQPLLAVDVTTQSQGEKPEEDLLLQDTTMEDDNSANGEAWEFTETELETLGLEELEIDDDEESSDSDSLSVYDILKEWEESSKKE

SEQ ID NO: 5 - protein, 913 aa, Arabidopsis thaliana
MSLSHLLRRLCTTTTTTRSPLSISFLHQRIHNISLSPANEDPETTTGNNQDSEKYPNLNPIPNDPSQFQIPQNHTPPIPYPPIPHRTMAFSSAEEAAAERRRRKRRLRIEPPLHALRRDPSAPPPKRDPNAPRLPDSTSALVGQRLNLHNRVQSLIRASDLDAASKLARQSVFSNTRPTVFTCNAIIAAMYRAKRYSESISLFQYFFKQSNIVPNVVSYNQIINAHCDEGNVDEALEVYRHILANAPFAPSSVTYRHLTKGLVQAGRIGDAASLLREMLSKGQAADSTVYNNLIRGYLDLGDFDKAVEFFDELKSKCTVYDGIVNATFMEYWFEKGNDKEAMESYRSLLDKKFRMHPPTGNVLLEVFLKFGKKDEAWALFNEMLDNHAPPNILSVNSDTVGIMVNECFKMGEFSEAINTFKKVGSKVTSKPFVMDYLGYCNIVTRFCEQGMLTEAERFFAEGVSRSLPADAPSHRAMIDAYLKAERIDDAVKMLDRMVDVNLRVVADFGARVFGELIKNGKLTESAEVLTKMGEREPKPDPSIYDVVVRGLCDGDALDQAKDIVGEMIRHNVGVTTVLREFIIEVFEKAGRREEIEKILNSVARPVRNAGQSGNTPPRVPAVFGTTPAAPQQPRDRAPWTSQGVVHSNSGWANGTAGQTAGGAYKANNGQNPSWSNTSDNQQQQSWSNQTAGQQPPSWSRQAPGYQQQQSWSQQSGWSSPSGHQQSWTNQTAGQQQPWANQTPGQQQQWANQTPGQQQQLANQTPGQQQQWANQTPGQQQQWANQNNGHQQPWANQNTGHQQSWANQTPSQQQPWANQTTGQQQGWGNQTTGQQQQWANQTAGQQSGWTAQQQWSNQTASHQQSQWLNPVPGEVANQTPWSNSVDSHLPQQQEPGPSHECQETQEKKVVELRN

SEQ ID NO: 6 - protein, 196 aa, Flabovacterium okeianocoites
ALVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 7 - DNA, 5303 bp, Artificial Sequence
  p63-VP64
cgccattctgcctggggacgtcggagcaagcttgatttaggtgacactatagaatacaagctacttgttctttttgcaagatctccaccatggactataaggaccacgacggagactacaaggatcatgatattgattacaaagacgatgacgataagatggccccaaagaagaagcggaaggtcggtatccccgggggcgaagtgctgtataggacactgctggccaactgcgtggctgctgggaacgtgaagaagtccgaactggtcttcaacaagatgaaggatctggggttccccctgagcggctttacctgtgaccaaatgctgctgctgcacaaaaggattgatagaaagaaaatcgctgatgtcctgctgctgatggaaaaggaaaatatcaagccaagcctgctgacctacaagatcctgatcgatgtgaagggcgccaccaacgacattagcgggatggaacagattctggaaacaatgaaagacgagggcgtggagctggatttccaaacacaggccctgacagccaggcattactccggcgctggactgaaagataaggcagaaaaggtgctgaaggaaatggagggagagtccctggaagcaaataggagggcctttaaggacctgctgtccatttacgcctccctgggcagagaagacgaagtgaaaagaatttggaagatttgcgagtccaaaccatactttgaggaatccctggccgctatccaagcattcggcaagctgaataaggtgcaagaagccgaggcaatcttcgaaaagattgtgaagatggatagaagagcaagctccagcacatactccgtcctgctgagagtgtacgtggatcataagatgctgagcaaaggcaaagacctggtgaagagaatggccgagagcgggtgcagaattgaagccaccacctgggacgctctgatcaaactgtatgtcgaggctggggaggtggaaaaagccgattccctgctggataaagccagcaaacaatcccacactaaactgatgatgaatagcttcatgtatatcatggacgagtatagcaagaggggcgacgtgcacaataccgaaaaaatctttctgaaaatgagggaagccgggtatactagcggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagttgatctagattctgcagccctatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaac

SEQ ID NO: 8 - DNA, 5948 bp, Artificial Sequence
  pTac2-VP64
cgccattctgcctggggacgtcggagcaagcttgatttaggtgacactatagaatacaagctacttgttctttttgcaagatctccaccatggactataaggaccacgacggagactacaaggatcatgatattgattacaaagacgatgacgataagatggccccaaagaagaagcggaaggtcggtatccccgggtccctgaacgactttgcactggtctttaaggaattcgcaggaaggggggattggcaaagatccctgagactgtttaagtatatgcagaggcaaatctggtgcaaacccaatgagcatatctataccattatgatttccctgctggggagagaaggactgctggataaatgtctggaagtgtttgacgaaatgccttcccaaggagtgagcaggagcgtgttcagctacactgcactgattaacgcctacggcagaaacggcaggtacgaaactagcctggagctgctggacagaatgaaaaacgagaagatcagcccaagcatcctgacttataacacagtgatcaatgcttgtgccagaggcggactggactgggagggcctgctgggcctgttcgcagagatgaggcacgaagggattcaacccgacatcgtgacttacaatactctgctgtccgcatgtgcaattaggggcctgggggacgaagctgaaatggtcttcaggactatgaatgacggcggaatcgtgcccgatctgaccacatattcccatctggtcgagacctttgggaaactgaggagactggagaaggtgtgcgatctgctgggagaaatggctagcggaggctccctgccagatattacctcctacaacgtgctgctggaagcctacgccaagtccggctccatcaaggaggctatgggcgtgtttcatcagatgcaagccgctggctgtacccccaatgccaacacctattccgtcctgctgaatctgttcggccagagcgggagatacgatgacgtgaggcagctgtttctggaaatgaagagcagcaacaccgaccccgacgctgcaacatacaacattctgatcgaggtgtttggcgaggggggctacttcaaagaagtcgtcaccctgttccacgacatggtggaggaaaacatcgagcccgatatggagacctatgaggggatcatcttcgcttgcggcaaaggcggcctgcatgaggacgctaggaagatcctgcagtacatgaccgctaatgacattgtcccatcctccaaagcttataccggcgtgatcgaggccttcggccaggctgccctgtacgaggaagcactggtcgcctttaacaccatgcacgaggtcggcagcaacccttccatcgagaccttccactccctgctgtatagcttcgccagaggcgggctggtgaaggagtccgaggcaatcctgagcaggctggtcgattccggcatccccaggaacagagacacctttaatgctcaaattgaggcctacaaacagggggggaagttcgaagaggctgtgaagacctacgtcgacatggaaaagagcaggtgcgaccccgacgagaggaccctggaggccgtcctgtccgtgtattccttcgcaagactggtggatgagtgcagggaacagtttgaagaaatgaaggccagcgacattctgcccagcattatgtgctactgcatgatgctggcagtgtacgggaagaccgagaggtgggacgacgtgaacgaactgctggaggagatgctgagcaacagggtcagcaacggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagttgatctagattctgcagccctatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaac

SEQ ID NO: 9 - DNA, 5531 bp, Artificial Sequence
  GUN1-VP64
cgccattctgcctggggacgtcggagcaagcttgatttaggtgacactatagaatacaagctacttgttctttttgcaagatctccaccatggactataaggaccacgacggagactacaaggatcatgatattgattacaaagacgatgacgataagatggccccaaagaagaagcggaaggtcggtatccccgggcaaggcaagctggcaagcgccatgatctccaccctgggcaggtacggaaaggtgaccattgccaagaggatcttcgagaccgccttcgcaggcgggtacggcaacaccgtgtatgctttttccgccctgattagcgcatatggcagaagcggcctgcacgaagaggccattagcgtgtttaacagcatgaaggagtatggactgaggcccaacctggtgacctacaacgccgtcattgatgcttgcggcaagggcggcatggaattcaagcaggtggccaagttcttcgatgaaatgcagaggaacggcgtgcagcctgacagaattacattcaatagcctgctggctgtgtgcagcagagggggcctgtgggaggcagctaggaatctgtttgacgagatgaccaatagaaggatcgagcaggacgtgttctcctataatacactgctggacgccatttgtaaaggcgggcaaatggacctggccttcgaaatcctggcccagatgcccgtcaaaaggatcatgcccaacgtggtcagctactccacagtcatcgacgggttcgccaaggctggcaggtttgatgaagcactgaacctgttcggggaaatgagatacctgggaatcgccctggacagggtgagctacaacaccctgctgagcatctacactaaggtcggcagatccgaggaagccctggacatcctgagggaaatggcctccgtgggcattaagaaggacgtcgtgacatacaatgccctgctgggcggctacggcaaacagggcaagtacgacgaggtcaagaaggtcttcacagagatgaagagggaacacgtgctgccaaatctgctgacttattccactctgattgatggctactccaaaggcggactgtacaaggaagccatggagattttcagagagttcaagagcgctggcctgagagccgatgtcgtgctgtattccgcactgatcgatgcactgtgcaaaaacggcctggtcggcagcgccgtgagcctgatcgacgagatgaccaaggagggaattagccccaatgtggtgacttacaatagcatcattgatgctttcggcagaagcgccaccatggacagatccgccgactatagcaacggcggcagcctgccattttcctccagcgccctgggatccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagttgatctagattctgcagccctatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaattcgcggccgcggcgccaatgcattgggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagtcgaccatagccaattcaatatggcgtatatggactcatgccaattcaatatggtggatctggacctgtgccaattcaatatggcgtatatggactcgtgccaattcaatatggtggatctggaccccagccaattcaatatggcggacttggcaccatgccaattcaatatggcggacttggcactgtgccaactggggaggggtctacttggcacggtgccaagtttgaggaggggtcttggccctgtgccaagtccgccatattgaattggcatggtgccaataatggcggccatattggctatatgccaggatcaatatataggcaatatccaatatggccctatgccaatatggctattggccaggttcaatactatgtattggccctatgccatatagtattccatatatgggttttcctattgacgtagatagcccctcccaatgggcggtcccatataccatatatggggcttcctaataccgcccatagccactcccccattgacgtcaatggtctctatatatggtctttcctattgacgtcatatgggcggtcctattgacgtatatggcgcctcccccattgacgtcaattacggtaaatggcccgcctggctcaatgcccattgacgtcaataggaccacccaccattgacgtcaatgggatggctcattgcccattcatatccgttctcacgccccctattgacgtcaatgacggtaaatggcccacttggcagtacatcaatatctattaatagtaacttggcaagtacattactattggaaggacgccagggtacattggcagtactcccattgacgtcaatggcggtaaatggcccgcgatggctgccaagtacatccccattgacgtcaatggggaggggcaatgacgcaaatgggcgttccattgacgtaaatgggcggtaggcgtgcctaatgggaggtctatataagcaatgctcgtttagggaac

SEQ ID NO: 10 - DNA, 5135 bp, Artificial Sequence
  pminCMV-luc2
ggtaccgagctcttacgcgtgctagcccgggctcgagatctgatatcaagcttactagtgtcgaggtaggcgtgtacggtgggaggcctatataagcagagctcgtttagtgaaccgtcagatcgcctggaggtaccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattcttaactgcagttaatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 11 - DNA, 9 bp, Artificial Sequence
  Target sequence in p63
tctatcact

SEQ ID NO: 12 - DNA, 15 bp, Artificial Sequence
  Target sequence in pTac2
aactttcgtcactca

SEQ ID NO: 13 - DNA, 11 bp, Artificial Sequence
  Target sequence in GUN1
aatttgtcgat

SEQ ID NO: 14 - DNA, 5204 bp, Artificial Sequence
  p63-4x target
ggtaccgagctcttacgcgtgctagcccgggctcgagatctgatatcaagttctatcactttgtttcttctatcacttgaattcttctatcacttatcttcttctatcacttcagttcgcttactagtgtcgaggtaggcgtgtacggtgggaggcctatataagcagagctcgtttagtgaaccgtcagatcgcctggaggtaccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattcttaactgcagttaatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 15 - DNA, 5272 bp, Artificial Sequence
  p63-8x target
ggtaccgagctcttacgcgtgctagcccgggctcgagatctgatatcaagttctatcacttccatggttctatcacttcacgacttctatcactttgtttcttctatcacttgaattcttctatcacttaagttcttctatcacttttcgaattctatcacttatcttcttctatcacttcagttcgcttactagtgtcgaggtaggcgtgtacggtgggaggcctatataagcagagctcgtttagtgaaccgtcagatcgcctggaggtaccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattcttaactgcagttaatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 16 - DNA, 5228 bp, Artificial Sequence
  pTac2-4x target
ggtaccgagctcttacgcgtgctagcccgggctcgagatctgatatcaagtaactttcgtcactcattgtttctaactttcgtcactcatggattctaactttcgtcactcatatcttctaactttcgtcactcatcagttcgcttactagtgtcgaggtaggcgtgtacggtgggaggcctatataagcagagctcgtttagtgaaccgtcagatcgcctggaggtaccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattcttaactgcagttaatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 17 - DNA, 5320 bp, Artificial Sequence
  pTac2-8x target
ggtaccgagctcttacgcgtgctagcccgggctcgagatctgatatcaagtaactttcgtcactcatccatggtaactttcgtcactcatcacgactaactttcgtcactcattgtttctaactttcgtcactcatgaattctaactttcgtcactcataagttctaactttcgtcactcatttcgaataactttcgtcactcatatcttctaactttcgtcactcatcagttcgcttactagtgtcgaggtaggcgtgtacggtgggaggcctatataagcagagctcgtttagtgaaccgtcagatcgcctggaggtaccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattcttaactgcagttaatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 18 - DNA, 5212 bp, Artificial Sequence
  GUN1-4x target
ggtaccgagctcttacgcgtgctagcccgggctcgagatctgatatcaagtaatttgtcgatttgtttctaatttgtcgattgaattctaatttgtcgattatcttctaatttgtcgattcagttcgcttactagtgtcgaggtaggcgtgtacggtgggaggcctatataagcagagctcgtttagtgaaccgtcagatcgcctggaggtaccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattcttaactgcagttaatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 19 - DNA, 5288 bp, Artificial Sequence
  GUN1-8x target
ggtaccgagctcttacgcgtgctagcccgggctcgagatctgatatcaagtaatttgtcgattccatggtaatttgtcgattcacgactaatttgtcgatttgtttctaatttgtcgattgaattctaatttgtcgattaagttctaatttgtcgattttcgaataatttgtcgattatcttctaatttgtcgattcagttcgcttactagtgtcgaggtaggcgtgtacggtgggaggcctatataagcagagctcgtttagtgaaccgtcagatcgcctggaggtaccgccaccatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgaattcttaactgcagttaatctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatctgaacgatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgata

SEQ ID NO: 20 - protein, 35 aa, Arabidopsis thaliana
VTYNTLISGLCKAGRLEEALELFEEMKEKGIAPDV

SEQ ID NO: 21 - protein, 31 aa, Arabidopsis thaliana
VVYNALIDMYAKCGDLEEARKVFDEMPERDV

SEQ ID NO: 22 - protein, 35 aa, Arabidopsis thaliana
FTLASVLKACASLGALSLGKQIHGYVIKSGFDSDE

SEQ ID NO: 23 - protein, 36 aa, Arabidopsis thaliana
VTFLGVLSACSHSGLVEEGLEYFESMKEKYGIEPDE

(19) United States
(12) Patent Application Publication
(10) Pub. No.: US 2022/0064229 A1
(43) Pub. Date: Mar. 3, 2022

(54) METHODS FOR DESIGNING DNA BINDING PROTEIN CONTAINING PPR MOTIFS, AND USE THEREOF

(71) Applicants: KYUSHU UNIVERSITY, NAT'L UNIVERSITY CORPORATION, Fukuoka-shi (JP); HIROSHIMA UNIVERSITY, Higashihiroshima-Shi (JP)

(72) Inventors: Takashi Yamamoto, Higashihiroshima-shi (JP); Tetsushi Sakuma, Higashihiroshima-shi (JP); Takahiro Nakamura, Fukuoka-shi (JP); Yusuke Yagi, Fukuoka-shi (JP); Yasuyuki Okawa, Fukuoka-shi (JP)

(73) Assignees: KYUSHU UNIVERSITY, NAT'L UNIVERSITY CORPORATION, Fukuoka-shi (JP); HIROSHIMA UNIVERSITY, Higashihiroshima-Shi (JP)

(21) Appl. No.: 17/365,292

(22) Filed: Jul. 1, 2021

Related U.S. Application Data
(63) Continuation-in-part of application No. 16/216,617, filed on Dec. 11, 2018, now abandoned.
Application No. 16/216,617 is a division of application No. 14/785,952, filed on Oct. 21, 2015, now Pat. No. 10,189,879.

(30) Foreign Application Priority Data
Apr. 22, 2013 (JP) 2013-089840

(51) Int. Cl. C07K 14/415; C12N 9/22; C12N 15/82; C12N 15/85

(52) U.S. Cl. CPC C07K 14/415; C12N 9/22; C12N 15/8213; C12Y 301/21004; C12N 15/8217; C12N 15/85; C07K 2319/80; C12N 15/8216

(57) ABSTRACT

A method for designing a protein capable of binding in a DNA base selective manner or DNA base sequence specific manner is provided. According to the present invention, it was revealed that, with a protein that can bind in a DNA base-selective manner or a DNA base sequence-specific manner, which contains one or more, preferably 2 to 30, more preferably 5 to 25, most preferably 9 to 15, of PPR motifs having a structure of the following formula 1 (wherein, in the formula 1, Helix A is a part that can form an α-helix structure; X does not exist, or is a part consisting of 1 to 9 amino acids; Helix B is a part that can form an α-helix structure; and L is a part consisting of 2 to 7 amino acids), and having a specific combination of amino acids corresponding to a DNA base or DNA base sequence as amino acids of three positions of No. 1 A.A., No. 4 A.A., in Helix A of the formula 1 and No. “ii” (-2) A.A. contained in L of the formula 1, the aforementioned object could be achieved.

(Helix A)-X-(Helix B)-L (Formula 1)

### CROSS-REFERENCE OF RELATED APPLICATIONS

This application is a Continuation-in-part of application Ser. No. 16/216,617, filed Dec. 11, 2018, which is a Divisional of application Ser. No. 14/785,952, filed Oct. 21, 2015, which is a 371 of International Application No. PCT/JP2014/061329, filed Apr. 22, 2014, which claims priority of Japanese Patent Application No. 2013-089840, filed Apr. 22, 2013, the entire contents of which are incorporated herein by reference.

### TECHNICAL FIELD

The present invention relates to a method for designing a DNA binding proteins containing PPR motifs and use thereof. According to the present invention, a pentatricopeptide repeat (PPR) motif is utilized. The present invention can be used for identification and design of a DNA-binding protein, identification of a target DNA of a protein having a PPR motif, and functional control of DNA. The present invention is useful in the fields of medicine, agricultural science, and so forth. The present invention also relates to a novel DNA-cleaving enzyme that utilizes a complex of a protein containing a PPR motif and a protein that defines a functional region.

### BACKGROUND ART

In recent years, techniques of binding nucleic acid-binding protein factors elucidated through various analyses to an intended sequence have been established, and they are coming to be used. Use of this sequence-specific binding is enabling analysis of intracellular localization of a target nucleic acid (DNA or RNA), elimination of a target DNA sequence, or expression control (activation or inactivation) of a protein-encoding gene existing downstream of a target DNA sequence. There are being conducted researches and developments using the zinc finger protein (Non-patent documents 1 and 2), TAL effecter (TALE, Non-patent document 3, Patent document 1), and CRISPR (Non-patent documents 4 and 5) as protein factors that act on DNA as materials for protein engineering. However, types of such protein factors are still extremely limited.

For example, the artificial enzyme, zinc finger nuclease (ZFN), known as an artificial DNA-cleaving enzyme, is a chimera protein obtained by binding a part that is constituted by linking 3 to 6 zinc fingers that specifically recognize a DNA consisting of 3 or 4 nucleotides and bind to it, and recognizes a nucleotide sequence in a sequence unit of 3 or 4 nucleotides with one DNA cleavage domain of a bacterial DNA-cleaving enzyme (for example, FokI) (Non-patent document 2). In such a chimera protein, the zinc finger domain is a protein domain that is known to bind to DNA, and it is based on the knowledge that many transcription factors have the aforementioned domain, and bind to a specific DNA sequence to control expression of a gene. By using two of ZFNs each having three zinc fingers, cleavage of one site per 70 billion nucleotides can be induced in theory.

However, because of the high cost required for the production of ZFNs, etc., the methods using ZFNs have not come to be widely used yet. Moreover, functional sorting efficiency of ZFNs is bad, and it is suggested that the methods have a problem also in this respect. Furthermore, since a zinc finger domain consisting of n of zinc fingers tends to recognize a sequence of (GNN)n, the methods also have a problem that degree of freedom for the target gene sequence is low.

An artificial enzyme, TALEN, has also been developed by binding a protein consisting of a combinatory sequence of module parts that can recognize every one nucleotide, TAL effecter (TALE), with a DNA cleavage domain of a bacterial DNA-cleaving enzyme (for example, FokI), and it is being investigated as an artificial enzyme that can replace ZFNs (Non-patent document 3). This TALEN is an enzyme generated by fusing a DNA binding domain of a transcription factor of a plant pathogenic Xanthomonas bacterium, and the DNA cleavage domain of the DNA restriction enzyme FokI, and it is known to bind to a neighboring DNA sequence to form a dimer and cleave a double strand DNA. Since, as for this molecule, the DNA binding domain of TALE found from a bacterium that infects with plants recognize one base with a combination of amino acids at two sites in the TALE motif consisting of 34 amino acid residues, it has a characteristic that binding property for a target DNA can be chosen by choosing the repetitive structure of the TALE module. TALEN using the DNA binding domain that has such a characteristic as mentioned above has a characteristic that it enables introduction of mutation into a target gene, like ZFNs, but the significant superiority thereof to ZFNs is that degree of freedom for the target gene (nucleotide sequence) is markedly improved, and the nucleotide to which it binds can be defined with a code.

However, since the total conformation of TALEN has not been elucidated, the DNA cleavage site of TALEN has not been identified at present. Therefore, it has a problem that cleavage site of TALEN is inaccurate, and is not fixed, compared with ZFNs, and it also cleaves even a similar sequence. Therefore, it has a problem that a nucleotide sequence cannot be accurately cleaved at an intended target site with a DNA-cleaving enzyme. For these reasons, it is desired to develop and provide a novel artificial DNA-cleaving enzyme free from the aforementioned problems.

On the basis of genome sequence information, PPR proteins (proteins having a pentatricopeptide repeat (PPR) motif) constituting a big family of no less than 500 members only for plants have been identified (Non-patent document 6). The PPR proteins are nucleus-encoded proteins, but are known to act on or involved in control, cleavage, translation, splicing, RNA edition, and RNA stability chiefly at an RNA level in organelles (chloroplasts and mitochondria) in a gene-specific manner. The PPR proteins typically have a structure consisting of about 10 contiguous 35-amino acid motifs of low conservativeness, i.e., PPR motifs, and it is considered that the combination of the PPR motifs is responsible for the sequence-selective binding with RNA. Almost all the PPR proteins consist only of repetition of about 10 PPR motifs, and any domain required for exhibiting a catalytic action is not found in many cases. Therefore, it is considered that the PPR proteins are essentially RNA adapters (Non-patent document 7).

In general, binding of a protein and DNA, and binding of a protein and RNA are attained by different molecular mechanisms. Therefore, a DNA-binding protein generally does not bind to RNA, whereas an RNA-binding protein generally does not bind to DNA. For example, in the case of the pumilio protein, which is known as an RNA-binding factor, and can encode RNA to be recognized, binding thereof to DNA has not been reported (Non-patent documents 8 and 9).

However, in the process of investigating properties of various kinds of PPR proteins, it became clear that it could be suggested that some types of the PPR proteins worked as DNA-binding factors.

The wheat p63 is a PPR protein having 9 PPR motifs, and it is suggested by gel shift assay that it binds to DNA in a sequence-specific manner (Non-patent document 10).

The GUN1 protein of Arabidopsis thaliana has 11 PPR motifs, and it is suggested by pull down assay that it binds with DNA (Non-patent document 11).

It has been demonstrated by run-on assay that the Arabidopsis thaliana pTac2 (protein having 15 PPR motifs, Non-patent document 12) and Arabidopsis thaliana DG1 (protein having 10 PPR motifs, Non-patent document 12) directly participate in transcription for generating RNA by using DNA as a template, and they are considered to bind to DNA.

An Arabidopsis thaliana strain deficient in the gene of GRP23 (protein having 11 PPR motifs, Non-patent document 14) shows the phenotype of embryonal death. It has been demonstrated that this protein physically interacts with the major subunit of the eukaryotic RNA transcription polymerase 2, which is a DNA-dependent RNA transcription enzyme, and therefore it is considered that GRP23 also acts to bind to DNA.

However, bindings of these PPR proteins to DNA have been only indirectly suggested, and actual sequence-specific binding has not been fully verified. Moreover, even if such proteins bind with DNA, it is generally considered that binding of a protein and DNA, and binding of a protein and RNA are attained by different molecular mechanisms, and therefore what kind of sequence rule specifically exists, with which binding is attained, etc., are not even expected at all.

### PRIOR ART REFERENCES

### Patent Documents

Patent document 1: WO2011/072246
Patent document 2: WO2011/111829

### Non-Patent Documents

Non-patent document 1: Maeder, M. L., et al. (2008) Rapid “open-source” engineering of customized zinc-finger nucleases for highly efficient gene modification, Mol. Cell 31, 294-301
Non-patent document 2: Urnov, F. D., et al. (2010) Genome editing with engineered zinc finger nucleases, Nature Review Genetics, 11, 636-646
Non-patent document 3: Miller, J. C., et al. (2011) A TALE nuclease architecture for efficient genome editing, Nature Biotech., 29, 143-148
Non-patent document 4: Mali P., et al. (2013) RNA-guided human genome engineering via Cas9, Science, 339, 823-826
Non-patent document 5: Cong L., et al. (2013) Multiplex genome engineering using CRISPR/Cas systems, Science, 339, 819-823
Non-patent document 6: Small, I. D. and Peeters, N. (2000) The PPR motif—a TPR-related motif prevalent in plant organellar proteins, Trends Biochem. Sci., 25, 46-47
Non-patent document 7: Woodson, J. D., and Chory, J. (2008) Coordination of gene expression between organellar and nuclear genomes, Nature Rev. Genet., 9, 383-395
Non-patent document 8: Wang, X., et al. (2002) Modular recognition of RNA by a human pumilio-homology domain, Cell, 110, 501-512
Non-patent document 9: Cheong, C. G, and Hall and T. M. (2006) Engineering RNA sequence specificity of Pumilio repeats, Proc. Natl. Acad. Sci. USA 103, 13635-13639
Non-patent document 10: Ikeda T. M. and Gray M. W. (1999) Characterization of a DNA-binding protein implicated in transcription in wheat mitochondria, Mol. Cell Bio., 119 (12): 8113-8122
Non-patent document 11: Koussevitzky S., et al. (2007) Signals from chloroplasts converge to regulate nuclear gene expression, Science, 316:715-719
Non-patent Document 12: Pfalz J, et al. (2006) PTAC2, -6, and -12 are components of the transcriptionally active plastid chromosome that are required for plastid gene expression, Plant Cell 18:176-197
Non-patent document 13: Chi W, et al. (2008) The pentatricopeptide repeat protein DELAYED GREENING1 is involved in the regulation of early chloroplast development and chloroplast gene expression in Arabidopsis, Plant Physiol., 147:573-584
Non-patent document 14: Ding Y H, et al. (2006) Arabidopsis GLUTAMINE-RICH PROTEIN 23 is essential for early embryogenesis and encodes a novel nuclear PPR motif protein that interacts with RNA polymerase II subunit III, Plant Cell, 18:815-830

### SUMMARY OF THE INVENTION

### Object to be Achieved by the Invention

The inventors of the present invention expected that the properties of the PPR proteins (proteins having a PPR motif) as RNA adapters would be determined by property of each PPR motif constituting the PPR proteins and combination of a plurality of PPR motifs, and proposed methods for modifying RNA-binding proteins using such PPR motifs (Patent document 2). Then, they elucidated that a PPR motif and RNA bind in one-to-one correspondence, contiguous PPR motifs recognize contiguous RNA bases in an RNA sequence, and such RNA recognition is determined by combination of amino acids at specific three positions among the 35 amino acids constituting the PPR motif, and filed a patent application for a method for designing a customized RNA-binding protein utilizing RNA recognition codes of PPR motifs and use thereof (PCT/JP2012/077274; Yagi, Y., et al. (2013) PLoS One, 8, e57286; and Barkan, A., et al. (2012) PLoS Genet., 8, e1002910).

It has been generally considered that binding of a protein and DNA, and binding of a protein and RNA are attained by different molecular mechanisms. However, the inventors of the present invention predicted that the RNA recognition rule of the PPR motif would be also usable for recognition of DNA, and analyzed PPR proteins that act to bind with DNA aiming at retrieving PPR proteins having such a characteristic. They also aimed at providing a novel artificial enzyme by preparing a customized DNA-binding protein that binds to a desired sequence using such a PPR protein that specifically binds to a DNA obtained as described above, and using it with a protein that defines a functional region, and providing a novel artificial DNA-cleaving enzyme by using it together with a region having a DNA-cleaving activity as the functional region.

### Means for Achieving the Object

As for the PPR proteins, it was elucidated by various domain search programs (Pfam, Prosite, Interpro, etc.) that the PPR motifs contained in the common RNA-binding type PPR proteins and the PPR motifs contained in the DNA-binding PPR proteins of some kinds mentioned above are not particularly distinguished. Therefore, it was considered that PPR proteins might contain amino acids (amino acid group) that would determine a binding property for DNA or a binding property for RNA apart from the amino acids required for the nucleic acid recognition.

The inventors of the present invention elucidated that an RNA-binding PPR motif and RNA bind in one-to-one correspondence, contiguous PPR motifs recognize contiguous RNA bases in an RNA sequence, and in such recognition, base-selective binding with RNA is determined by combination of RNA recognition amino acids at specific three positions (that is, the first and fourth amino acids of the first helix (Helix A) among the two α-helix structures constituting the motif (No. 1 A.A. and No. 4 A.A.), and the second amino acid counted from the C-terminus (No. “ii” (-2) A.A.)), among the 35 amino acids constituting the PPR motif, and filed a patent application for a method for designing a customized RNA-binding protein utilizing RNA recognition codes of PPR motifs and use thereof (PCT/JP2012/077274).

Then, among the PPR proteins, for the aforementioned wheat p63 (Non-patent document 11, the amino acid sequence of the homologous protein of Arabidopsis thaliana is shown as SEQ ID NO: 1), GUN1 protein of Arabidopsis thaliana (Non-patent document 12, amino acid sequence thereof is shown as SEQ ID NO: 2), pTac2 of Arabidopsis thaliana (Non-patent document 13, amino acid sequence thereof is shown as SEQ ID NO: 3), DG1 (Non-patent document 14, amino acid sequence thereof is shown as SEQ ID NO: 4), and GRP23 of Arabidopsis thaliana (Non-patent document 15, amino acid sequence thereof is shown as SEQ ID NO: 5), for which binding with DNA was suggested, amino acid frequencies of the amino acids at three positions bearing the nucleic acid recognition codes in the PPR motif considered to be important when RNA is a target (No. 1 A.A., No. 4 A.A. and No. “ii” (-2) A.A.) were compared with those found in the RNA binding type motif. As a result, it became clear that the tendencies of the amino acid frequencies found in those PPR motifs as mentioned above, for which DNA-binding property was suggested, and the RNA binding type motifs substantially agreed with each other.

The above results suggest that the nucleic acid recognition codes of the RNA binding type PPR motifs can also be applied to the DNA binding type PPR motifs. Thymine (T) is a uracil (U) derivative having a structure consisting of uracil (U) of which carbon of the 5-position is methylated, as it is also called 5-methyluracil. Such a characteristic of the base constituting the nucleic acid suggests that the combination of the amino acids that recognizes uracil (U) of an RNA binding type PPR motif is used for recognition of thymine (T) in DNA.

On the basis of the aforementioned findings, it was elucidated that, by using the aforementioned p63 (amino acid sequence of SEQ ID NO: 1), GUN1 protein of Arabidopsis thaliana (amino acid sequence of SEQ ID NO: 2), pTac2 of Arabidopsis thaliana (amino acid sequence of SEQ ID NO: 3), DG1 (amino acid sequence of SEQ ID NO: 4), and GRP23 of Arabidopsis thaliana (amino acid sequence of SEQ ID NO: 5), which are DNA-binding type PPR proteins, as a template, arranging amino acids of the three positions (No. 1 A.A., No. 4 A.A. and No. “ii” (-2) A.A.) with applying the finding obtained for such PPR proteins as a result of examination of the RNA-binding type PPR motifs, a customized DNA-binding protein that binds to an arbitrary DNA base sequence could be produced.

That is, the inventors of the present invention provided a protein that comprises 2 or more, preferably 2 to 30, more preferably 5 to 25, most preferably 9 to 15, of PPR motifs having the specific amino acids described later as the amino acids at the three positions (No. 1 A.A., No. 4 A.A., and No. “ii” (-2) A.A.) in the PPR motifs, and can bind to DNA in a DNA base-selective manner or DNA base sequence-selective manner, of which typical examples are the amino acid sequences of SEQ ID NOS: 1 to 5, and thus accomplished the present invention.

The present invention provides the followings.

[1] A method for designing a DNA-binding protein that can bind in a DNA base-selective manner or a DNA base sequence-specific manner, the method including: determining an amino acid sequence of the DNA-binding protein, wherein the DNA-binding protein contains one or more motifs having a structure of the following formula 1.

[Formula 1]

(Helix A)-X-(Helix B)-L (Formula 1)

(wherein, in the formula 1:
Helix A is a part that can form an α-helix structure;
X does not exist, or is a part consisting of 1 to 9 amino acids;
Helix B is a part that can form an α-helix structure; and
L is a part consisting of 2 to 7 amino acids),
wherein,
under the following definitions:
the first amino acid of Helix A is referred to as No. 1 amino acid (No. 1 A.A.), the fourth amino acid as No. 4 amino acid (No. 4 A.A.), and
when a next PPR motif (Mₙ₊₁) contiguously exists on the C-terminus side of the PPR motif (Mₙ) (when there is no amino acid insertion between the PPR motifs), the −2nd amino acid counted from the end (C-terminus side) of the amino acids constituting the PPR motif (Mₙ);
when a non-PPR motif consisting of 1 to 20 amino acids exists between the PPR motif (Mₙ) and the next PPR motif (Mₙ₊₁) on the C-terminus side, the amino acid locating upstream of the first amino acid of the next PPR motif (Mₙ₊₁) by 2 positions, i.e., the −2nd amino acid; or
when any next PPR motif (Mₙ₊₁) does not exist on the C-terminus side of the PPR motif (Mₙ), or 21 or more amino acids constituting a non-PPR motif exist between the PPR motif (Mₙ) and the next PPR motif (Mₙ₊₁) on the C-terminus side, the 2nd amino acid counted from the end (C-terminus side) of the amino acids constituting the PPR motif (Mₙ)
   is referred to as No. “ii” (-2) amino acid (No. “ii” (-2) A.A.),
   one PPR motif (Mₙ) contained in the protein is a PPR motif having a specific combination of amino acids corresponding to a target DNA base or target DNA base sequence as the three amino acids of No. 1 A.A., No. 4 A.A., and No. “ii” (-2) A.A.
   [2] The protein according to [1], wherein the combination of the three amino acids of No. 1 A.A., No. 4 A.A., and No. “ii” (-2) A.A. is a combination corresponding to a target DNA base or target DNA base sequence, and the combination of amino acids is determined according to any one of the following definitions:
   (1-1) when No. 4 A.A. is glycine (G), No. 1 A.A. may be an arbitrary amino acid, and No. “ii” (-2) A.A. is aspartic acid (D), asparagine (N), or serine (S);
   (1-2) when No. 4 A.A. is isoleucine (I), each of No. 1 A.A. and No. “ii” (-2) A.A. may be an arbitrary amino acid;
   (1-3) when No. 4 A.A. is leucine (L), each of No. 1 A.A. and No. “ii” (-2) A.A. may be an arbitrary amino acid;
   (1-4) when No. 4 A.A. is methionine (M), each of No. 1 A.A. and No. “ii” (-2) A.A. may be an arbitrary amino acid;
   (1-5) when No. 4 A.A. is asparagine (N), each of No. 1 A.A. and No. “ii” (-2) A.A. may be an arbitrary amino acid;
   (1-6) when No. 4 A.A. is proline (P), each of No. 1 A.A. and No. “ii” (-2) A.A. may be an arbitrary amino acid;
   (1-7) when No. 4 A.A. is serine (S), each of No. 1 A.A. and No. “ii” (-2) A.A. may be an arbitrary amino acid;
   (1-8) when No. 4 A.A. is threonine (T), each of No. 1 A.A. and No. “ii” (-2) A.A. may be an arbitrary amino acid; and
   (1-9) when No. 4 A.A. is valine (V), each of No. 1 A.A. and No. “ii” (-2) A.A. may be an arbitrary amino acid.
   [3] The protein according to [1], wherein the combination of the three amino acids of No. 1 A.A., No. 4 A.A., and No. “ii” (-2) A.A. is a combination corresponding to a target DNA base or target DNA base sequence, and the combination of amino acids is determined according to any one of the following definitions:
   (2-1) when the three amino acids, No. 1 A.A., No. 4 A.A., and No. “ii” (-2) A.A. are an arbitrary amino acid, glycine, and aspartic acid, respectively, the PPR motif selectively binds to G;
   (2-2) when the three amino acids, No. 1 A.A., No. 4 A.A., and No. “ii” (-2) A.A., are glutamic acid, glycine, and aspartic acid, respectively, the PPR motif selectively binds to G;
   (2-3) when the three amino acids, No. 1 A.A., No. 4 A.A., and No. “ii” (-2) A.A., are an arbitrary amino acid, glycine, and asparagine, respectively, the PPR motif selectively binds to A;
   (2-4) when the three amino acids, No. 1 A.A., No. 4 A.A., and No. “ii” (-2) A.A., are glutamic acid, glycine, and asparagine, respectively, the PPR motif selectively binds to A;
   (2-5) when the three amino acids, No. 1 A.A., No. 4 A.A., and No. “ii” (-2) A.A., are an arbitrary amino acid, glycine, and serine, respectively, the PPR motif selectively binds to A, and next binds to C;
   (2-6) when the three amino acids, No. 1 A.A., No. 4 A.A., and No. “ii” (-2) A.A., are an arbitrary amino acid, isoleucine, and an arbitrary amino acid, respectively, the PPR motif selectively binds to T and C;
   (2-7) when the three amino acids, No. 1 A.A., No. 4 A.A., and No. “ii” (-2) A.A., are an arbitrary amino acid, isoleucine, and asparagine, respectively, the PPR motif selectively binds to T, and next binds to C;
   (2-8) when the three amino acids, No. 1 A.A., No. 4 A.A., and No. “ii” (-2) A.A., are an arbitrary amino acid, leucine, and an arbitrary amino acid, respectively, the PPR motif selectively binds to T and C;
   (2-9) when the three amino acids, No. 1 A.A., No. 4 A.A., and No. “ii” (-2) A.A., are an arbitrary amino acid, leucine, and aspartic acid, respectively, the PPR motif selectively binds to C;
   (2-10) when the three amino acids, No. 1 A.A., No. 4 A.A., and No. “ii” (-2) A.A., are an arbitrary amino acid, leucine, and lysine, respectively, the PPR motif selectively binds to T;
   (2-11) when the three amino acids, No. 1 A.A., No. 4 A.A., and No. “ii” (-2) A.A., are an arbitrary amino acid, methionine, and an arbitrary amino acid, respectively, the PPR motif selectively binds to T;
   (2-12) when the three amino acids, No. 1 A.A., No. 4 A.A., and No. “ii” (-2) A.A., are an arbitrary amino acid, methionine, and aspartic acid, respectively, the PPR motif selectively binds to T;
   (2-13) when the three amino acids, No. 1 A.A., No. 4 A.A., and No. “ii” (-2) A.A., are isoleucine, methionine, and aspartic acid, respectively, the PPR motif selectively binds to T, and next binds to C;
   (2-14) when the three amino acids, No. 1 A.A., No. 4 A.A., and No. “ii” (-2) A.A., are an arbitrary amino acid, asparagine, and an arbitrary amino acid, respectively, the PPR motif selectively binds to C and T;
   (2-15) when the three amino acids, No. 1 A.A., No. 4 A.A., and No. “ii” (-2) A.A., are an arbitrary amino acid, asparagine, and aspartic acid, respectively, the PPR motif selectively binds to T;
   (2-16) when the three amino acids, No. 1 A.A., No. 4 A.A., and No. “ii” (-2) A.A., are phenylalanine, asparagine, and aspartic acid, respectively, the PPR motif selectively binds to T;
   (2-17) when the three amino acids, No. 1 A.A., No. 4 A.A., and No. “ii” (-2) A.A., are glycine, asparagine, and aspartic acid, respectively, the PPR motif selectively binds to T;
   (2-18) when the three amino acids, No. 1 A.A., No. 4 A.A., and No. “ii” (-2) A.A., are isoleucine, asparagine, and aspartic acid, respectively, the PPR motif selectively binds to T;
   (2-19) when the three amino acids, No. 1 A.A., No. 4 A.A., and No. “ii” (-2) A.A., are threonine, asparagine, and aspartic acid, respectively, the PPR motif selectively binds to T;
   (2-20) when the three amino acids, No. 1 A.A., No. 4 A.A., and No. “ii” (-2) A.A. are valine, asparagine, and aspartic acid, respectively, the PPR motif selectively binds to T, and next binds to C;
   (2-21) when the three amino acids, No. 1 A.A., No. 4 A.A., and No. “ii” (-2) A.A. are tyrosine, asparagine, and aspartic acid, respectively, the PPR motif selectively binds to T, and next binds to C;
   (2-22) when the three amino acids, No. 1 A.A., No. 4 A.A., and No. “ii” (-2) A.A., are an arbitrary amino acid, asparagine, and asparagine, respectively, the PPR motif selectively binds to C;
   (2-23) when the three amino acids, No. 1 A.A., No. 4 A.A., and No. “ii” (-2) A.A., are isoleucine, asparagine, and asparagine, respectively, the PPR motif selectively binds to C;
   (2-24) when the three amino acids, No. 1 A.A., No. 4 A.A., and No. “ii” (-2) A.A., are serine, asparagine, and asparagine, respectively, the PPR motif selectively binds to C;
   (2-25) when the three amino acids, No. 1 A.A., No. 4 A.A., and No. “ii” (-2) A.A., are valine, asparagine, and asparagine, respectively, the PPR motif selectively binds to C;
   (2-26) when the three amino acids, No. 1 A.A., No. 4 A.A., and No. “ii” (-2) A.A., are an arbitrary amino acid, asparagine, and serine, respectively, the PPR motif selectively binds to C;
   (2-27) when the three amino acids, No. 1 A.A., No. 4 A.A., and No. “ii” (-2) A.A., are valine, asparagine, and serine, respectively, the PPR motif selectively binds to C;
   (2-28) when the three amino acids, No. 1 A.A., No. 4 A.A., and No. “ii” (-2) A.A., are an arbitrary amino acid, asparagine, and threonine, respectively, the PPR motif selectively binds to C;
   (2-29) when the three amino acids, No. 1 A.A., No. 4 A.A., and No. “ii” (-2) A.A., are valine, asparagine, and threonine, respectively, the PPR motif selectively binds to C;
   (2-30) when the three amino acids, No. 1 A.A., No. 4 A.A., and No. “ii” (-2) A.A., are an arbitrary amino acid, asparagine, and tryptophan, respectively, the PPR motif selectively binds to C, and next binds to T;
   (2-31) when the three amino acids, No. 1 A.A., No. 4 A.A., and No. “ii” (-2) A.A., are isoleucine, asparagine, and tryptophan, respectively, the PPR motif selectively binds to T, and next binds to C;
   (2-32) when the three amino acids, No. 1 A.A., No. 4 A.A., and No. “ii” (-2) A.A., are an arbitrary amino acid, proline, and an arbitrary amino acid, respectively, the PPR motif selectively binds to T;
   (2-33) when the three amino acids, No. 1 A.A., No. 4 A.A., and No. “ii” (-2) A.A., are an arbitrary amino acid, proline, and aspartic acid, respectively, the PPR motif selectively binds to T;
   (2-34) when the three amino acids, No. 1 A.A., No. 4 A.A., and No. “ii” (-2) A.A., are phenylalanine, proline, and aspartic acid, respectively, the PPR motif selectively binds to T;
   (2-35) when the three amino acids, No. 1 A.A., No. 4 A.A., and No. “ii” (-2) A.A., are tyrosine, proline, and aspartic acid, respectively, the PPR motif selectively binds to T;
   (2-36) when the three amino acids, No. 1 A.A., No. 4 A.A., and No. “ii” (-2) A.A., are an arbitrary amino acid, serine, and an arbitrary amino acid, respectively, the PPR motif selectively binds to A and G;
   (2-37) when the three amino acids, No. 1 A.A., No. 4 A.A., and No. “ii” (-2) A.A., are an arbitrary amino acid, serine, and asparagine, respectively, the PPR motif selectively binds to A;
   (2-38) when the three amino acids, No. 1 A.A., No. 4 A.A., and No. “ii” (-2) A.A., are phenylalanine, serine, and asparagine, respectively, the PPR motif selectively binds to A;
   (2-39) when the three amino acids, No. 1 A.A., No. 4 A.A., and No. “ii” (-2) A.A., are valine, serine, and asparagine, respectively, the PPR motif selectively binds to A;
   (2-40) when the three amino acids, No. 1 A.A., No. 4 A.A., and No. “ii” (-2) A.A., are an arbitrary amino acid, threonine, and an arbitrary amino acid, respectively, the PPR motif selectively binds to A and G;
   (2-41) when the three amino acids, No. 1 A.A., No. 4 A.A., and No. “ii” (-2) A.A., are an arbitrary amino acid, threonine, and aspartic acid, respectively, the PPR motif selectively binds to G;
   (2-42) when the three amino acids, No. 1 A.A., No. 4 A.A., and No. “ii” (-2) A.A., are valine, threonine, and aspartic acid, respectively, the PPR motif selectively binds to G;
   (2-43) when the three amino acids, No. 1 A.A., No. 4 A.A., and No. “ii” (-2) A.A., are an arbitrary amino acid, threonine, and asparagine, respectively, the PPR motif selectively binds to A;
   (2-44) when the three amino acids, No. 1 A.A., No. 4 A.A., and No. “ii” (-2) A.A., are phenylalanine, threonine, and asparagine, respectively, the PPR motif selectively binds to A;
   (2-45) when the three amino acids, No. 1 A.A., No. 4 A.A., and No. “ii” (-2) A.A., are isoleucine, threonine, and asparagine, respectively, the PPR motif selectively binds to A;
   (2-46) when the three amino acids, No. 1 A.A., No. 4 A.A., and No. “ii” (-2) A.A., are valine, threonine, and asparagine, respectively, the PPR motif selectively binds to A;
   (2-47) when the three amino acids, No. 1 A.A., No. 4 A.A., and No. “ii” (-2) A.A., are an arbitrary amino acid, valine, and an arbitrary amino acid, respectively, the PPR motif binds with A, C, and T, but does not bind to G;
   (2-48) when the three amino acids, No. 1 A.A., No. 4 A.A., and No. “ii” (-2) A.A., are isoleucine, valine, and aspartic acid, respectively, the PPR motif selectively binds to C, and next binds to A;
   (2-49) when the three amino acids, No. 1 A.A., No. 4 A.A., and No. “ii” (-2) A.A., are an arbitrary amino acid, valine, and glycine, respectively, the PPR motif selectively binds to C; and
   (2-50) when the three amino acids, No. 1 A.A., No. 4 A.A., and No. “ii” (-2) A.A., are an arbitrary amino acid, valine, and threonine, respectively, the PPR motif selectively binds to T.
   [4] The protein according to any one of [1] to [3], which contains 2 to 30 of the PPR motifs (Mₙ) defined in [1].
   [5] The protein according to any one of [1] to [3], which contains 5 to 25 of the PPR motifs (Mₙ) defined in [1].
   [6] The protein according to any one of [1] to [3], which contains 9 to 15 of the PPR motifs (Mₙ) defined in [1].
   [7] The PPR protein according to [6], which consists of a sequence selected from the amino acid sequence of SEQ ID NO: 1 containing 9 PPR motifs, the amino acid sequence of SEQ ID NO: 2 containing 11 PPR motifs, the amino acid sequence of SEQ ID NO: 3 containing 15 PPR motifs, the amino acid sequence of SEQ ID NO: 4 containing 10 PPR motifs, and the amino acid sequence of SEQ ID NO: 5 containing 11 PPR motifs.
   [8] A method for identifying a DNA base or DNA base sequence that serves as a target of a DNA-binding protein containing one or more (preferably 2 to 30) PPR motifs (Mₙ) defined in [1], wherein:

the DNA base or DNA base sequence is identified by determining presence or absence of a DNA base corresponding to a combination of the three amino acids of No. 1 A.A., No. 4 A.A., and No. “ii” (-2) A.A. of the PPR motif on the basis of any one of the definitions (1-1) to (1-9) mentioned in [2], and (2-1) to (2-50) mentioned in [3].

[9] A method for identifying a PPR protein containing one or more (preferably 2 to 30) PPR motifs (Mₙ) defined in [1] that can bind to a target DNA base or target DNA having a specific base sequence, wherein:

the PPR protein is identified by determining presence or absence of a combination of the three amino acids of No. 1 A.A., No. 4 A.A., and No. “ii” (-2) A.A. corresponding to the target DNA base or a specific base constituting the target DNA on the basis of any one of the definitions (1-1) to (1-9) mentioned in [2], and (2-1) to (2-50) mentioned in [3].

[10] A method for controlling a function of DNA, which uses the protein according to [1].
[11] A complex consisting of a region comprising the protein according to [1], and a functional region bound together.
[12] The complex according to [11], wherein the functional region is fused to the protein according to [1] on the C-terminus side of the protein.
[13] The complex according to [11] or [12], wherein the functional region is a DNA-cleaving enzyme, or a nuclease domain thereof, or a transcription control domain, and the complex functions as a target sequence-specific DNA-cleaving enzyme or transcription control factor.
[14] The complex according to [13], wherein the DNA-cleaving enzyme is the nuclease domain of FokI (SEQ ID NO: 6).
[15] A method for modifying a genetic substance of a cell comprising the following steps:

preparing a cell containing a DNA having a target sequence; and

introducing the complex according to [11] into the cell so that the region of the complex consisting of the protein binds to the DNA having a target sequence, and therefore the functional region modifies the DNA having a target sequence.

[16] A method for identifying, recognizing, or targeting a DNA base or DNA having a specific base sequence by using a PPR protein containing one or more PPR motifs.
[17] The method according to [16], wherein the protein contains one or more PPR motifs in which three amino acids among the amino acids constituting the motif constitute a specific combination of amino acids.
[18] The method according to [16] or [17], wherein the protein contains one or more PPR motifs (Mₙ) defined in [1].

### Effect of the Invention

According to the present invention, a PPR motif that can binds to a target DNA base, and a protein containing it can be provided. By arranging two or more PPR motifs, a protein that can binds to a target DNA having an arbitrary sequence or length can be provided.

According to the present invention, a target DNA of an arbitrary PPR protein can be predicted and identified, and conversely, a PPR protein that binds to an arbitrary DNA can be predicted and identified. Prediction of such a target DNA sequence clarifies the genetic identity thereof, and increases possibility of use thereof. Furthermore, according to the present invention, functionalities of homologous genes of a gene of an industrially useful PPR protein showing amino acid polymorphism at a high level can be determined on the basis of difference of the target DNA base sequences thereof.

Furthermore, according to the present invention, a novel DNA-cleaving enzyme using a PPR motif can also be provided. That is, by linking a protein as a functional region with the PPR motif or PPR protein provided by the present invention, a complex containing a protein having a binding activity for a specific nucleic acid sequence, and a protein having a specific functionality can be prepared.

The functional region usable in the present invention is one that can impart, among various functions, a function for any one of cleavage, transcription, replication, restoration, synthesis, modification, etc. of DNA. By choosing the sequence of the PPR motifs, which is the characteristic of the present invention, to determine a base sequence of DNA as a target, almost all DNA sequences can be used as a target, and genome edition using a function of the functional region such as those for cleavage, transcription, replication, restoration, synthesis, modification, etc. of DNA can be realized with such a target.

For example, when the functional region has a function for cleaving DNA, a complex comprising a PPR protein part prepared according to the present invention and a DNA-cleaving region linked together is provided. Such a complex can function as an artificial DNA-cleaving enzyme, which recognizes a base sequence of DNA as a target with the PPR protein part, and then cleaves DNA with the region for cleaving DNA. When the functional region has a transcription control function, a complex comprising a PPR protein part prepared according to the present invention and a transcription control region for DNA linked together is provided. Such a complex can function as an artificial transcription control factor, which recognizes a base sequence of DNA as a target with the PPR protein part, and then promotes transcription of the target DNA.

The present invention can further be utilized for a method for delivering the aforementioned complex in a living body so that the complex functions in the living body, and preparation of transformants utilizing a nucleic acid sequence (DNA and RNA) encoding a protein obtained according to the present invention, as well as specific modification, control, and impartation of a function in various situations in organisms (cells, tissues, and individuals).

### BRIEF DESCRIPTION OF THE DRAWINGS

FIGS. 1A-1C show conserved sequences and amino acid numbers of the PPR motif. FIG. 1A shows the amino acids constituting the PPR motif defined in the present invention, and the amino acid numbers thereof (the amino acid sequences P, S, L1, and L2 correspond to SEQ ID NO: 20, SEQ ID NO: 21, SEQ ID NO: 22, and SEQ ID NO: 23, respectively). FIG. 1B shows positions of three amino acids (No. 1 A.A., No. 4 A.A., and No. “ii” (-2) A.A.) that control binding base selectivity in the predicted structure. FIG. 1C shows two examples of the structure of the PPR motif, and the positions of the amino acids on the predicted structure for each case. No. 1 A.A., No. 4 A.A., and No. “ii” (-2) A.A. are indicated with sticks of magenta color (dark gray in the case of monochratic display) in the conformational diagrams of the protein.

FIG. 2 summarizes the outlines of the structures of Arabidopsis thaliana p63 (amino acid sequence of SEQ ID NO: 1), the GUN1 protein of Arabidopsis thaliana (amino acid sequence of SEQ ID NO: 2), pTac2 of Arabidopsis thaliana (amino acid sequence of SEQ ID NO: 3), DG1 (amino acid sequences of SEQ ID NO: 4), and GRP23 of Arabidopsis thaliana (amino acid sequence of SEQ ID NO: 5), which are DNA-binding type PPR proteins that function in DNA metabolism, and the outline of the assay system for demonstrating that they bind to DNA.

FIG. 3 summarizes the amino acid frequencies of the amino acids at the three positions bearing the nucleic acid recognition codes in the PPR motif (No. 1 A.A., No. 4 A.A., and No. “ii” (-2) A.A.) for the PPR motifs of the PPR proteins (SEQ ID NOS: 1 to 5), for which DNA binding property was suggested, and known RNA-binding type motifs.

FIG. 4-1 shows the positions of the PPR motifs included in the inside of the proteins, and the positions of the three amino acids bearing the nucleic acid recognition codes (No. 1 A.A., No. 4 A.A., and No. “ii” (-2) A.A.) in the PPR motifs for each of (A) Arabidopsis thaliana p63 (amino acid sequence of SEQ ID NO: 1) and (B) the GUN1 protein of Arabidopsis thaliana (amino acid sequence of SEQ ID NO: 2.

FIG. 4-2 shows the positions of the PPR motifs included in the inside of the proteins, and the positions of the three amino acids bearing the nucleic acid recognition codes (No. 1 A.A., No. 4 A.A., and No. “ii” (-2) A.A.) in the PPR motifs for each of (C) pTac2 of Arabidopsis thaliana (amino acid sequence of SEQ ID NO: 3), and (D) DG1 (amino acid sequence of SEQ ID NO: 4).

FIG. 4-3 shows the positions of the PPR motifs included in the inside of the proteins, and the positions of the three amino acids bearing the nucleic acid recognition codes (No. 1 A.A., No. 4 A.A., and No. “ii” (-2) A.A.) in the PPR motifs for (E) GRP23 of Arabidopsis thaliana (amino acid sequence of SEQ ID NO: 5).

FIG. 5 shows the evaluation of the sequence-specific DNA-binding abilities of the PPR molecules. Artificial transcription factors were prepared by fusing each of three kinds of DNA-binding type (regarded so) PPR molecules with VP64, which is a transcription activation domain, and whether they could activate a luciferase reporter having each target sequence was examined in a human cultured cell.

FIG. 6 shows comparison of the luciferase activities observed by cointroduction of pTac2-VP64 or GUN1-VP64 with pminCMV-luc2 as a negative control, or a reporter vector comprising 4 or 8 target sequences. As a result, there was observed a tendency that the activity increased with increase of the target sequence for the both molecules, and thus it was verified that these PPR-VP64 molecules specifically bound to each target sequence to function as a site-specific transcription activator.

### MODES FOR CARRYING OUT THE INVENTION

[PPR Motif and PPR Protein]

The “PPR motif” referred to in the present invention means a polypeptide constituted with 30 to 38 amino acids and having an amino acid sequence that shows, when the amino acid sequence is analyzed with a protein domain search program on the web (for example, Pfam, Prosite, Uniprot, etc.), an E value not larger than a predetermined value (desirably E-03) obtained at PF01535 in the case of Pfam (http://pfam.sanger.ac.uk/), or PS51375 in the case of Prosite (http://www.expasy.org/prosite/), unless otherwise indicated. The PPR motifs in various proteins are also defined in the Uniprot database (http://www.uniprot.org).

Although the amino acid sequence of the PPR motif is not highly conserved in the PPR motif of the present invention, such a secondary structure of helix, loop, helix, and loop as shown by the following formula is conserved well.

[Formula 2]

(Helix A)-X-(Helix B)-L (Formula 1)

The position numbers of the amino acids constituting the PPR motif defined in the present invention are according to those defined in a paper of the inventors of the present invention (Kobayashi K, et al., Nucleic Acids Res., 40, 2712-2723 (2012)). That is, the position numbers of the amino acids constituting the PPR motif defined in the present invention are substantially the same as the amino acid numbers defined for PF01535 in Pfam, but correspond to numbers obtained by subtracting 2 from the amino acid numbers defined for PS51375 in Prosite (for example, position 1 according to the present invention is position 3 of PS51375), and also correspond to numbers obtained by subtracting 2 from the amino acid numbers of the PPR motif defined in Uniprot.

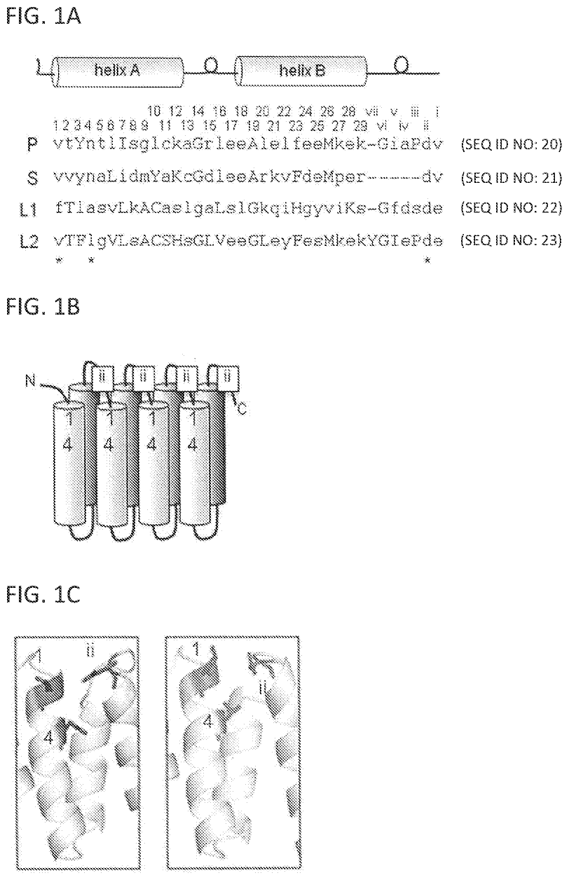

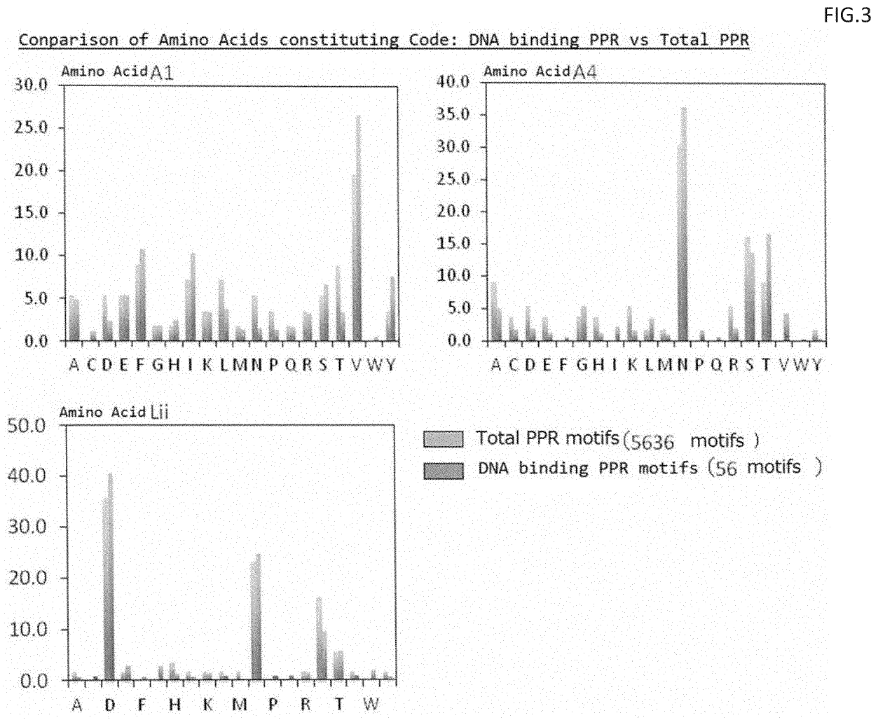

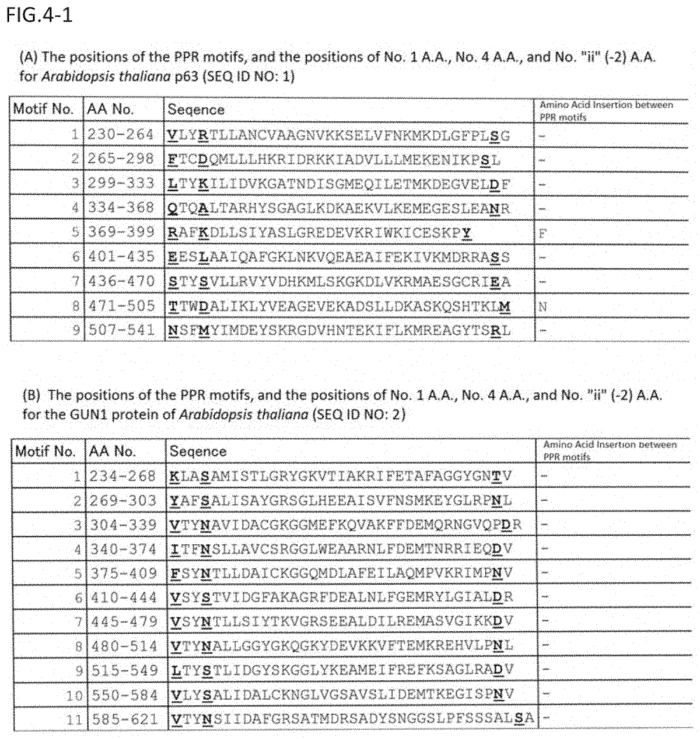

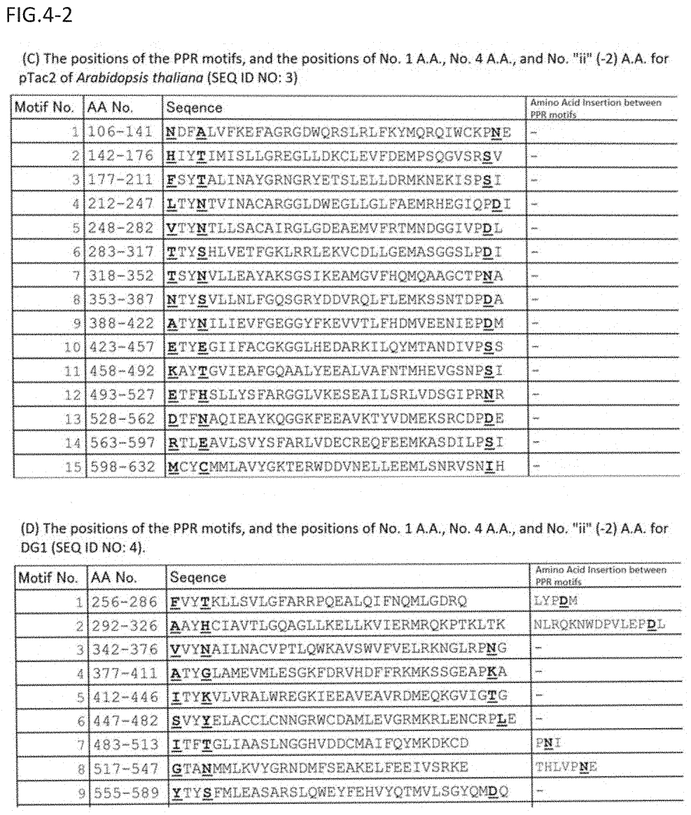

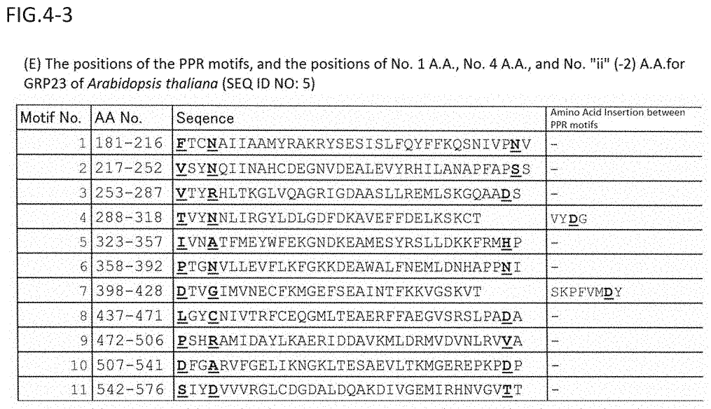

More precisely, in the present invention, the No. 1 amino acid is the first amino acid from which Helix A shown in the formula 1 starts. The No. 4 amino acid is the fourth amino acid counted from the No. 1 amino acid. As for “ii” (-2)nd amino acid,
when a next PPR motif (Mₙ₊₁) contiguously exists on the C-terminus side of the PPR motif (Mₙ) (when there is no amino acid insertion between the PPR motifs, as in the cases of, for example, Motif Nos. 1, 2, 3, 4, 6 and 7 in FIG. 4-1 (A)), the −2nd amino acid counted from the end (C-terminus side) of the amino acids constituting the PPR motif (Mₙ) is referred to as No. “ii” (-2) amino acid;
when a non-PPR motif (part that is not the PPR motif) consisting of 1 to 20 amino acids exists between the PPR motif (Mₙ) and the next PPR motif (Mₙ₊₁) on the C-terminus side (as in the cases of, for example, Motif Nos. 5 and 8 in FIG. 4-1 (A), and Motif Nos. 1, 2, 7 and 8 in FIG. 4-3 (D)), the amino acid locating upstream of the first amino acid of the next PPR motif (Mₙ₊₁) by 2 positions, i.e., the −2nd amino acid, is referred to as No. “ii” (-2) amino acid (refer to FIG. 1); or
when any next PPR motif (Mₙ₊₁) does not exist on the C-terminus side of the PPR motif (Mₙ) (as in the cases of, for example, Motif No. 9 in FIG. 4-1 (A), and Motif No. 11 in FIG. 4-1 (B)), or 21 or more amino acids constituting a non-PPR motif exist between the PPR motif (Mₙ) and the next PPR motif (Mₙ₊₁) on the C-terminus side, the 2nd amino acid counted from the end (C-terminus side) of the amino acids constituting the PPR motif (Mₙ) is referred to as No. “ii” (-2) amino acid.

The “PPR protein” referred to in the present invention means a PPR protein having two or more of the aforementioned PPR motifs, unless otherwise indicated. The term “protein” used in this specification means any substance consisting of a polypeptide (chain consisting of two or more amino acids bound through peptide bonds), and also includes those consisting of a comparatively low molecular weight polypeptide, unless otherwise indicated. The “amino acid” referred to in the present invention means a usual amino acid molecule, as well as an amino acid residue constituting a peptide chain. Which the term means will be apparent to those skilled in the art from the context.

Many PPR proteins exist in plants, and 500 proteins and about 5000 motifs can be found in Arabidopsis thaliana. PPR motifs and PPR proteins of various amino acid sequences also exist in many land plants such as rice, poplar, and selaginella. It is known that some PPR proteins are important factors for obtaining F1 seeds for hybrid vigor as fertility restoration factors that are involved in formation of pollen (male gamete). It has been clarified that some PPR proteins are involved in speciation, similarly in fertility restoration. It has also been clarified that almost all the PPR proteins act on RNA in mitochondria or chloroplasts.

It is known that, in animals, anomaly of the PPR protein identified as LRPPRC causes Leigh syndrome French Canadian (LSFC, Leigh's syndrome, subacute necrotizing encephalomyelopathy).

The term “selective” used for a property of a PPR motif for binding with a DNA base in the present invention means that a binding activity for any one base among the DNA bases is higher than binding activities for the other bases, unless otherwise indicates. Those skilled in the art can confirm this selectivity by planning an experiment, or it can also be obtained by calculation as described in the examples mentioned in this specification.

The DNA base referred to in the present invention means a base of deoxyribonucleotide constituting DNA, and specifically, it means any of adenine (A), guanine (G), cytosine (C), and thymine (T), unless otherwise indicated. Although the PPR protein may have selectivity to a base in DNA, it does not bind to a nucleic acid monomer.

Although search methods for conserved amino acid sequence as the PPR motif had been established before the present invention was accomplished, any rule concerning selective binding with DNA base had not been discovered at all.

[Findings Provided by the Present Invention]

The following findings are provided by the present invention.

(I) Information about Positions of Amino Acids Important for Selective Binding

Specifically, under the following definitions:

the first amino acid of Helix A of the PPR motif is referred to as No. 1 amino acid (No. 1 A.A.), the fourth amino acid as No. 4 amino acid (No. 4 A.A.), and
when a next PPR motif (Mₙ₊₁) contiguously exists on the C-terminus side of the PPR motif (Mₙ) (when there is no amino acid insertion between the PPR motifs), the −2nd amino acid counted from the end (C-terminus side) of the amino acids constituting the PPR motif (Mₙ);
when a non-PPR motif consisting of 1 to 20 amino acids exist between the PPR motif (Mₙ) and the next PPR motif (Mₙ₊₁) on the C-terminus side, the amino acid locating upstream of the first amino acid of the next PPR motif (Mₙ₊₁) by 2 positions, i.e., the −2nd amino acid; or
when any next PPR motif (Mₙ₊₁) does not exist on the C-terminus side of the PPR motif (Mₙ), or 21 or more amino acids constituting a non-PPR motif exist between the PPR motif (Mₙ) and the next PPR motif (Mₙ₊₁) on the C-terminus side, the 2nd amino acid counted from the end (C-terminus side) of the amino acids constituting the PPR motif (Mₙ)
   is referred to as No. “ii” (-2) amino acid (No. “ii” (-2) A.A.), combination of the three amino acids, the first and fourth amino acids of the helix (Helix A), No. 1 and No. 4 amino acids, and No. “ii” (-2) A.A. defined above (No. 1 A.A., No. 4 A.A. and No. “ii” (-2) A.A.) is important for selective binding to a DNA base, and to what kind of DNA base the motif binds can be determined on the basis of the combination.

The present invention is based on the findings concerning the combination of the three amino acids, No. 1 A.A., No. 4 A.A., and No. “ii” (-2) A.A., found by the inventors of the present invention. Specifically:

(1-1) when No. 4 A.A. is glycine (G), No. 1 A.A. may be an arbitrary amino acid, No. “ii” (-2) A.A. is aspartic acid (D), asparagine (N), or serine (S), and the combination of No. 1 A.A., and No. “ii” (-2) A.A. may be, for example:
a combination of an arbitrary amino acid and aspartic acid (D) (*GD),
preferably a combination of glutamic acid (E) and aspartic acid (D) (EGD),
a combination of an arbitrary amino acid and asparagine (N) (*GN),
preferably a combination of glutamic acid (E) and asparagine (N) (EGN), or
a combination of an arbitrary amino acid and serine (S) (*GS);
   (1-2) when No. 4 A.A. is isoleucine (I), each of No. 1 A.A. and No. “ii” (-2) A.A. may be an arbitrary amino acid, and the combination of No. 1 A.A., and No. “ii” (-2) A.A. may be, for example:
a combination of an arbitrary amino acid and asparagine (N) (*IN);
   (1-3) when No. 4 A.A. is leucine (L), each of No. 1 A.A. and No. “ii” (-2) A.A. may be an arbitrary amino acid, and the combination of No. 1 A.A., and No. “ii” (-2) A.A. may be, for example:
a combination of an arbitrary amino acid and aspartic acid (D) (*LD), or
a combination of an arbitrary amino acid and lysine (K) (*LK);
   (1-4) when No. 4 A.A. is methionine (M), each of No. 1 A.A. and No. “ii” (-2) A.A. may be an arbitrary amino acid, and the combination of No. 1 A.A., and No. “ii” (-2) A.A. may be, for example:
a combination of an arbitrary amino acid and aspartic acid (D) (*MD), or
a combination of isoleucine (I) and aspartic acid (D) (IMD);
   (1-5) when No. 4 A.A. is asparagine (N), each of No. 1 A.A. and No. “ii” (-2) A.A. may be an arbitrary amino acid, and the combination of No. 1 A.A., and No. “ii” (-2) A.A. may be, for example:
a combination of an arbitrary amino acid and aspartic acid (D) (*ND),
a combination of any one of phenylalanine (F), glycine (G), isoleucine (I), threonine (T), valine
   (V) and tyrosines (Y), and aspartic acid (D) (FND, GND, IND, TND, VND, or YND),
a combination of an arbitrary amino acid and asparagine (N) (*NN),
a combination of any one of isoleucine (I), serine (S) and valine (V), and asparagine (N) (INN, SNN or VNN)
a combination of an arbitrary amino acid and serine (S) (*NS),
a combination of valine (V) and serine (S) (VNS),
a combination of an arbitrary amino acid and threonine (T) (*NT),
a combination of valine (V) and threonine (T) (VNT),
a combination of an arbitrary amino acid and tryptophan (W) (*NW), or
a combination of isoleucine (I) and tryptophan (W) (INW);
   (1-6) when No. 4 A.A. is proline (P), each of No. 1 A.A. and No. “ii” (-2) A.A. may be an arbitrary amino acid, and the combination of No. 1 A.A., and No. “ii” (-2) A.A. may be, for example:
a combination of an arbitrary amino acid and aspartic acid (D) (*PD),
a combination of phenylalanine (F) and aspartic acid (D) (FPD), or
a combination of tyrosine (Y) and aspartic acid (D) (YPD);
   (1-7) when No. 4 A.A. is serine (S), each of No. 1 A.A. and No. “ii” (-2) A.A. may be an arbitrary amino acid, and the combination of No. 1 A.A., and No. “ii” (-2) A.A. may be, for example:
a combination of an arbitrary amino acid and asparagine (N) (*SN),
a combination of phenylalanine (F) and asparagine (N) (FSN), or
a combination of valine (V) and asparagine (N) (VSN);
   (1-8) when No. 4 A.A. is threonine (T), each of No. 1 A.A. and No. “ii” (-2) A.A. may be an arbitrary amino acid, and the combination of No. 1 A.A., and No. “ii” (-2) A.A. may be, for example:
a combination of an arbitrary amino acid and aspartic acid (D) (*TD),
a combination of valine (V) and aspartic acid (D) (VTD),
a combination of an arbitrary amino acid and asparagine (N) (*TN),
a combination of phenylalanine (F) and asparagine (N) (FTN),
a combination of isoleucine (I) and asparagine (N) (ITN), or
a combination of valine (V) and asparagine (N) (VTN); and
   (1-9) when No. 4 A.A. is valine (V), each of No. 1 A.A. and No. “ii” (-2) A.A. may be an arbitrary amino acid, and the combination of No. 1 A.A., and No. “ii” (-2) A.A. may be, for example:
a combination of isoleucine (I) and aspartic acid (D) (IVD),
a combination of an arbitrary amino acid and glycine (G) (*VG), or
a combination of an arbitrary amino acid and threonine (T) (*VT).
   (II) Information about Correspondence of Combination of Three Amino Acids of No. 1 A.A., No. 4 A.A., and No. “ii” (-2) A.A., and DNA Base

The protein is a protein determined on the basis of, specifically, the following definitions, and having a selective DNA base-binding property:

(2-1) when the three amino acids, No. 1 A.A., No. 4 A.A., and No. “ii” (-2) A.A., are an arbitrary amino acid, glycine, and aspartic acid, respectively, the PPR motif selectively binds to G;
(2-2) when the three amino acids, No. 1 A.A., No. 4 A.A., and No. “ii” (-2) A.A., are glutamic acid, glycine, and aspartic acid, respectively, the PPR motif selectively binds to G;
(2-3) when the three amino acids, No. 1 A.A., No. 4 A.A., and No. “ii” (-2) A.A., are an arbitrary amino acid, glycine, and asparagine, respectively, the PPR motif selectively binds to A;
(2-4) when the three amino acids, No. 1 A.A., No. 4 A.A., and No. “ii” (-2) A.A., are glutamic acid, glycine, and asparagine, respectively, the PPR motif selectively binds to A;
(2-5) when the three amino acids, No. 1 A.A., No. 4 A.A., and No. “ii” (-2) A.A., are an arbitrary amino acid, glycine, and serine, respectively, the PPR motif selectively binds to A, and next binds to C;
(2-6) when the three amino acids, No. 1 A.A., No. 4 A.A., and No. “ii” (-2) A.A., are an arbitrary amino acid, isoleucine, and an arbitrary amino acid, respectively, the PPR motif selectively binds to T and C;
(2-7) when the three amino acids, No. 1 A.A., No. 4 A.A., and No. “ii” (-2) A.A., are an arbitrary amino acid, isoleucine, and asparagine, respectively, the PPR motif selectively binds to T, and next binds to C;
(2-8) when the three amino acids, No. 1 A.A., No. 4 A.A., and No. “ii” (-2) A.A., are an arbitrary amino acid, leucine, and an arbitrary amino acid, respectively, the PPR motif selectively binds to T and C;
(2-9) when the three amino acids, No. 1 A.A., No. 4 A.A., and No. “ii” (-2) A.A., are an arbitrary amino acid, leucine, and aspartic acid, respectively, the PPR motif selectively binds to C;
(2-10) when the three amino acids, No. 1 A.A., No. 4 A.A., and No. “ii” (-2) A.A., are an arbitrary amino acid, leucine, and lysine, respectively, the PPR motif selectively binds to T;
(2-11) when the three amino acids, No. 1 A.A., No. 4 A.A., and No. “ii” (-2) A.A., are an arbitrary amino acid, methionine, and an arbitrary amino acid, respectively, the PPR motif selectively binds to T;
(2-12) when the three amino acids, No. 1 A.A., No. 4 A.A., and No. “ii” (-2) A.A., are an arbitrary amino acid, methionine, and aspartic acid, respectively, the PPR motif selectively binds to T;
(2-13) when the three amino acids, No. 1 A.A., No. 4 A.A., and No. “ii” (-2) A.A., are isoleucine, methionine, and aspartic acid, respectively, the PPR motif selectively binds to T, and next binds to C;
(2-14) when the three amino acids, No. 1 A.A., No. 4 A.A., and No. “ii” (-2) A.A., are an arbitrary amino acid, asparagine, and an arbitrary amino acid, respectively, the PPR motif selectively binds to C and T;
(2-15) when the three amino acids, No. 1 A.A., No. 4 A.A., and No. “ii” (-2) A.A., are an arbitrary amino acid, asparagine, and aspartic acid, respectively, the PPR motif selectively binds to T;
(2-16) when the three amino acids, No. 1 A.A., No. 4 A.A., and No. “ii” (-2) A.A., are phenylalanine, asparagine, and aspartic acid, respectively, the PPR motif selectively binds to T;
(2-17) when the three amino acids, No. 1 A.A., No. 4 A.A., and No. “ii” (-2) A.A., are glycine, asparagine, and aspartic acid, respectively, the PPR motif selectively binds to T;
(2-18) when the three amino acids, No. 1 A.A., No. 4 A.A., and No. “ii” (-2) A.A., are isoleucine, asparagine, and aspartic acid, respectively, the PPR motif selectively binds to T;
(2-19) when the three amino acids, No. 1 A.A., No. 4 A.A., and No. “ii” (-2) A.A., are threonine, asparagine, and aspartic acid, respectively, the PPR motif selectively binds to T;
(2-20) when the three amino acids, No. 1 A.A., No. 4 A.A., and No. “ii” (-2) A.A. are valine, asparagine, and aspartic acid, respectively, the PPR motif selectively binds to T, and next binds to C;
(2-21) when the three amino acids, No. 1 A.A., No. 4 A.A., and No. “ii” (-2) A.A. are tyrosine, asparagine, and aspartic acid, respectively, the PPR motif selectively binds to T, and next binds to C;
(2-22) when the three amino acids, No. 1 A.A., No. 4 A.A., and No. “ii” (-2) A.A., are an arbitrary amino acid, asparagine, and asparagine, respectively, the PPR motif selectively binds to C;
(2-23) when the three amino acids, No. 1 A.A., No. 4 A.A., and No. “ii” (-2) A.A., are isoleucine, asparagine, and asparagine, respectively, the PPR motif selectively binds to C;
(2-24) when the three amino acids, No. 1 A.A., No. 4 A.A., and No. “ii” (-2) A.A., are serine, asparagine, and asparagine, respectively, the PPR motif selectively binds to C;
(2-25) when the three amino acids, No. 1 A.A., No. 4 A.A., and No. “ii” (-2) A.A., are valine, asparagine, and asparagine, respectively, the PPR motif selectively binds to C;
(2-26) when the three amino acids, No. 1 A.A., No. 4 A.A., and No. “ii” (-2) A.A., are an arbitrary amino acid, asparagine, and serine, respectively, the PPR motif selectively binds to C;
(2-27) when the three amino acids, No. 1 A.A., No. 4 A.A., and No. “ii” (-2) A.A., are valine, asparagine, and serine, respectively, the PPR motif selectively binds to C;
(2-28) when the three amino acids, No. 1 A.A., No. 4 A.A., and No. “ii” (-2) A.A., are an arbitrary amino acid, asparagine, and threonine, respectively, the PPR motif selectively binds to C;
(2-29) when the three amino acids, No. 1 A.A., No. 4 A.A., and No. “ii” (-2) A.A., are valine, asparagine, and threonine, respectively, the PPR motif selectively binds to C;
(2-30) when the three amino acids, No. 1 A.A., No. 4 A.A., and No. “ii” (-2) A.A., are an arbitrary amino acid, asparagine, and tryptophan, respectively, the PPR motif selectively binds to C, and next binds to T;
(2-31) when the three amino acids, No. 1 A.A., No. 4 A.A., and No. “ii” (-2) A.A., are isoleucine, asparagine, and tryptophan, respectively, the PPR motif selectively binds to T, and next binds to C;
(2-32) when the three amino acids, No. 1 A.A., No. 4 A.A., and No. “ii” (-2) A.A., are an arbitrary amino acid, proline, and an arbitrary amino acid, respectively, the PPR motif selectively binds to T;
(2-33) when the three amino acids, No. 1 A.A., No. 4 A.A., and No. “ii” (-2) A.A., are an arbitrary amino acid, proline, and aspartic acid, respectively, the PPR motif selectively binds to T;
(2-34) when the three amino acids, No. 1 A.A., No. 4 A.A., and No. “ii” (-2) A.A., are phenylalanine, proline, and aspartic acid, respectively, the PPR motif selectively binds to T;
(2-35) when the three amino acids, No. 1 A.A., No. 4 A.A., and No. “ii” (-2) A.A., are tyrosine, proline, and aspartic acid, respectively, the PPR motif selectively binds to T;
(2-36) when the three amino acids, No. 1 A.A., No. 4 A.A., and No. “ii” (-2) A.A., are an arbitrary amino acid, serine, and an arbitrary amino acid, respectively, the PPR motif selectively binds to A and G;
(2-37) when the three amino acids, No. 1 A.A., No. 4 A.A., and No. “ii” (-2) A.A., are an arbitrary amino acid, serine, and asparagine, respectively, the PPR motif selectively binds to A;
(2-38) when the three amino acids, No. 1 A.A., No. 4 A.A., and No. “ii” (-2) A.A., are phenylalanine, serine, and asparagine, respectively, the PPR motif selectively binds to A;
(2-39) when the three amino acids, No. 1 A.A., No. 4 A.A., and No. “ii” (-2) A.A., are valine, serine, and asparagine, respectively, the PPR motif selectively binds to A;
(2-40) when the three amino acids, No. 1 A.A., No. 4 A.A., and No. “ii” (-2) A.A., are an arbitrary amino acid, threonine, and an arbitrary amino acid, respectively, the PPR motif selectively binds to A and G;
(2-41) when the three amino acids, No. 1 A.A., No. 4 A.A., and No. “ii” (-2) A.A., are an arbitrary amino acid, threonine, and aspartic acid, respectively, the PPR motif selectively binds to G;
(2-42) when the three amino acids, No. 1 A.A., No. 4 A.A., and No. “ii” (-2) A.A., are valine, threonine, and aspartic acid, respectively, the PPR motif selectively binds to G;
(2-43) when the three amino acids, No. 1 A.A., No. 4 A.A., and No. “ii” (-2) A.A., are an arbitrary amino acid, threonine, and asparagine, respectively, the PPR motif selectively binds to A;
(2-44) when the three amino acids, No. 1 A.A., No. 4 A.A., and No. “ii” (-2) A.A., are phenylalanine, threonine, and asparagine, respectively, the PPR motif selectively binds to A;
(2-45) when the three amino acids, No. 1 A.A., No. 4 A.A., and No. “ii” (-2) A.A., are isoleucine, threonine, and asparagine, respectively, the PPR motif selectively binds to A;
(2-46) when the three amino acids, No. 1 A.A., No. 4 A.A., and No. “ii” (-2) A.A., are valine, threonine, and asparagine, respectively, the PPR motif selectively binds to A;
(2-47) when the three amino acids, No. 1 A.A., No. 4 A.A., and No. “ii” (-2) A.A., are an arbitrary amino acid, valine, and an arbitrary amino acid, respectively, the PPR motif binds with A, C, and T, but does not bind to G;
(2-48) when the three amino acids, No. 1 A.A., No. 4 A.A., and No. “ii” (-2) A.A., are isoleucine, valine, and aspartic acid, respectively, the PPR motif selectively binds to C, and next binds to A;
(2-49) when the three amino acids, No. 1 A.A., No. 4 A.A., and No. “ii” (-2) A.A., are an arbitrary amino acid, valine, and glycine, respectively, the PPR motif selectively binds to C; and
(2-50) when the three amino acids, No. 1 A.A., No. 4 A.A., and No. “ii” (-2) A.A., are an arbitrary amino acid, valine, and threonine, respectively, the PPR motif selectively binds to T.

Combination of amino acids of specific positions and binding property with a DNA base can be confirmed by experiments. Experiments for such purposes include preparation of a PPR motif or a protein containing two or more PPR motifs, preparation of a substrate DNA, and binding property test (for example, gel shift assay). These experiments are well known to those skilled in the art, and as for more specific procedures and conditions, for example, Patent document 2 can be referred to.

[Use of PPR Motif and PPR Protein]

Identification and Design

One PPR motif recognizes a specific one kind of base of DNA, and two or more contiguous PPR motifs can recognize continuous bases in a DNA sequence. Further, according to the present invention, by appropriately choosing amino acids at specific positions, PPR motifs selective for each of A, T, and C can be chosen or designed, and a protein containing an appropriate continuation of such PPR motifs can recognize a corresponding specific sequence. Therefore, according to the present invention, a naturally occurring PPR protein that selectively binds to DNA having a specific base sequence can be predicted or identified, or conversely, DNA as a target of binding of a PPR protein can be predicted and identified. Prediction or identification of such a target is useful for clarifying genetic identity of the target, and is also useful from a viewpoint that such prediction or identification may expand applicability of the target.

Furthermore, according to the present invention, a PPR motif that can selectively bind to a desired DNA base, and a protein having two or more PPR motifs that can bind to a desired DNA in a sequence-specific manner can be designed. In such design, as for the part other than the amino acids at the important positions in the PPR motif, sequence information on PPR motifs of naturally occurring type in DNA-binding type PPR proteins such as those of SEQ ID NOS: 1 to 5 can be referred to. Further, the motif or protein may also be designed by using a motif or protein of naturally occurring type as a whole, and replacing only the amino acids of the corresponding positions. Although the number of repetitions of PPR motifs can be appropriately chosen according to a target sequence, it may be, for example, 2 or more, preferably 2 to 30, more preferably 5 to 25, most preferably 9 to 15.

In the designing, amino acids other than those of the combination of the amino acids of No. 1 A.A., No. 4 A.A., and No. “ii” (-2) A.A. may be taken into consideration. For example, selection of the amino acids of No. 8 and No. 12 described in Patent document 2 mentioned above may be important for exhibiting a DNA-binding activity. According to the researches of the inventors of the present invention, the No. 8 amino acid of a certain PPR motif and the No. 12 amino acid of the same PPR motif may cooperate in binding with DNA. The No. 8 amino acid may be a basic amino acid, preferably lysine, or an acidic amino acid, preferably aspartic acid, and the No. 12 amino acid may be a basic amino acid, neutral amino acid, or hydrophobic amino acid.

A designed motif or protein can be prepared by methods well known to those skilled in the art. That is, the present invention provides a PPR motif that selectively binds to a specific DNA base, and a PPR protein that specifically binds to DNA having a specific sequence, in which attention is paid to the combination of the amino acids of No. 1 A.A., No. 4 A.A., and No. “ii” (-2) A.A. Such a motif and protein can be prepared even in a comparatively large amount by methods well known to those skilled in the art, and such methods may comprise determining a nucleic acid sequence encoding a target motif or protein from the amino acid sequence of the target motif or protein, cloning it, and preparing a transformant that produces the target motif or protein.

Preparation of Complex and Use Thereof

The PPR motif or PPR protein provided by the present invention can be made into a complex by binding a functional region. The functional region generally refers to a part having such a function as a specific biological function exerted in a living body or cell, for example, enzymatic function, catalytic function, inhibitory function, promotion function, etc, or a function as a marker. Such a region consists of, for example, a protein, peptide, nucleic acid, physiologically active substance, or drug.

According to the present invention, by binding a functional region to the PPR protein, the target DNA sequence-binding function exerted by the PPR protein, and the function exerted by the functional region can be exhibited in combination. For example, if a protein having a DNA-cleaving function (for example, restriction enzyme such as FokI) or a nuclease domain thereof is used as the functional region, the complex can function as an artificial DNA-cleaving enzyme.

In order to produce such a complex, methods generally available in this technical field can be used, and there are known a method of synthesizing such a complex as one protein molecule, a method of separately synthesizing two or more members of proteins, and then combining them to form a complex, and so forth.

In the case of the method of synthesizing a complex as one protein molecule, for example, a protein complex can be designed so as to comprise a PPR protein and a cleaving enzyme bound to the C-terminus of the PPR protein via an amino acid linker, an expression vector structure for expressing the protein complex can be constructed, and the target complex can be expressed from the structure. As such a preparation method, the method described in Japanese Patent Application No. 2011-242250, and so forth can be used.

For binding the PPR protein and the functional region protein, any binding means known in this technical field may be used, including binding via an amino acid linker, binding utilizing specific affinity such as binding between avidin and biotin, binding utilizing another chemical linker, and so forth.

The functional region usable in the present invention refers to a region that can impart any one of various functions such as those for cleavage, transcription, replication, restoration, synthesis, or modification of DNA, and so forth. By choosing the sequence of the PPR motif to define a DNA base sequence as a target, which is the characteristic of the present invention, substantially any DNA sequence may be used as the target, and with such a target, genome edition utilizing the function of the functional region such as those for cleavage, transcription, replication, restoration, synthesis, or modification of DNA can be realized.

For example, when the function of the functional region is a DNA cleavage function, there is provided a complex comprising a PPR protein part prepared according to the present invention and a DNA cleavage region bound together. Such a complex can function as an artificial DNA-cleaving enzyme that recognizes a base sequence of DNA as a target by the PPR protein part, and then cleaves DNA by the DNA cleavage region.

An example of the functional region having a cleavage function usable for the present invention is a deoxyribonuclease (DNase), which functions as an endodeoxyribonuclease. As such a DNase, for example, endodeoxyribonucleases such as DNase A (e.g., bovine pancreatic ribonuclease A, PDB 2AAS), DNase H and DNase I, restriction enzymes derived from various bacteria (for example, FokI (SEQ ID NO: 6) etc.) and nuclease domains thereof can be used. Such a complex comprising a PPR protein and a functional region does not exist in the nature, and is novel.

When the function of the functional region is a transcription control function, there is provided a complex comprising a PPR protein part prepared according to the present invention and a DNA transcription control region bound together. Such a complex can function as an artificial transcription control factor, which recognizes a base sequence of DNA as a target by the PPR protein part, and then controls transcription of the target DNA.

The functional region having a transcription control function usable for the present invention may be a domain that activates transcription, or may be a domain that suppresses transcription. Examples of the transcription control domain include VP16, VP64, TA2, STAT-6, and p65. Such a complex comprising a PPR protein and a transcription control domain does not exist in the nature, and is novel.

Further, the complex obtainable according to the present invention may deliver a functional region in a living body or cell in a DNA sequence-specific manner, and allow it to function. It thereby makes it possible to perform modification or disruption in a DNA sequence-specific manner in a living body or cell, like protein complexes utilizing a zinc finger protein (Non-patent documents 1 and 2 mentioned above) or TAL effecter (Non-patent document 3 and Patent document 1 mentioned above), and thus it becomes possible to impart a novel function, i.e., function for cleavage of DNA and genome edition utilizing that function. Specifically, with a PPR protein comprising two or more PPR motifs that can bind with a specific base linked together, a specific DNA sequence can be recognized. Then, genome edition of the recognized DNA region can be realized by the functional region bound to the PPR protein using the function of the functional region.

Furthermore, by binding a drug to the PPR protein that binds to a DNA sequence in a DNA sequence-specific manner, the drug may be delivered to the neighborhood of the DNA sequence as the target. Therefore, the present invention provides a method for DNA sequence-specific delivery of a functional substance.

It has been clarified that the PPR protein used as a material in the present invention works to specify an edition position for DNA edition, and such a PPR motif having specific amino acids arranged at the positions of the residues of No. 1 A.A., No. 4 A.A., and No. “ii” (-2) A.A. recognizes a specific base on DNA, and then exhibits the DNA-binding activity thereof. On the basis of such a characteristic, a PPR protein of this type that has specific amino acids arranged at the positions of the residues of No. 1 A.A., No. 4 A.A., and No. “ii” (-2) A.A. can be expected to recognize a base on DNA specific to each PPR protein, and as a result, introduce base polymorphism, or to be used in a treatment of a disease or condition resulting from a base polymorphism, and in addition, it is considered that the combination of such a PPR protein with such another functional region as mentioned above contribute to modification or improvement of functions for realizing cleavage of DNA for genome edition.

Moreover, an exogenous DNA-cleaving enzyme can be fused to the C-terminus of the PPR protein. Alternatively, by improving binding DNA base selectivity of the PPR motif on the N-terminus side, a DNA sequence-specific DNA-cleaving enzyme can also be constituted. Moreover, such a complex to which a marker part such as GFP is bound can also be used for visualization of a desired DNA in vivo.

### EXAMPLES

### Example 1: Collection of PPR Proteins and Target Sequences Thereof Used for DNA Edition

By referring to the information provided in the prior art references (Non-patent documents 11 to 15), structures and functions of the p63 protein (SEQ ID NO: 1), GUN1 protein (SEQ ID NO: 2), pTac2 protein (SEQ ID NO: 3), DG1 protein (SEQ ID NO: 4), and GRP23 protein (SEQ ID NO: 5) were analyzed.

To the PPR motif structures in such proteins, amino acid numbers defined in the present invention were imparted together with the information of the Uniprot database (http://www.uniprot.org/). The PPR motifs contained in the five kinds of PPR proteins of Arabidopsis thaliana (SEQ ID NOS: 1 to 5) used for the experiment, and the amino acid numbers thereof are shown in FIG. 3.

Specifically, amino acid frequencies for the amino acids at the three positions (No. 1 A.A., No. 4 A.A., and No. “ii” (-2) A.A.) responsible for the nucleic acid recognition codes in the PPR motifs considered to be important at the time of targeting RNA in the aforementioned p63 protein (SEQ ID NO: 1), GUN1 protein (SEQ ID NO: 2), pTac2 protein (SEQ ID NO: 3), DG1 protein (SEQ ID NO: 4), and GRP23 protein (SEQ ID NO: 5) were compared with those of RNA-binding type motifs.

The p63 protein of Arabidopsis thaliana (SEQ ID NO: 1) has 9 PPR motifs, and the positions of the residues of No. 1 A.A., No. 4 A.A., and No. “ii” (-2) A.A. in the amino acid sequence are as summarized in the following table and FIG. 3.

TABLE 1

| | | | | Code | Base to be | | | |
|---|---|---|---|---|---|---|---|---|
| | | | | (1, 4, | bound (ratio) | | | |
| | A₁ | A₄ | Lᵢᵢ | ii) | A | C | G | T |
| PPR | 230, V | 233, R | 263, S | *R* | 0.25 | 0.07 | 0.06 | 0.62 |
| motif 1 | | | | | | | | |
| PPR | 265, F | 368, D | 297, S | *D* | 0.25 | 0.24 | 0.23 | 0.29 |
| motif 2 | | | | | | | | |
| PPR | 299, L | 302, K | 332, D | *KD | 0.20 | 0.18 | 0.28 | 0.34 |
| motif 3 | | | | | | | | |
| PPR | 334, Q | 337, A | 367, N | *AN | 0.45 | 0.18 | 0.05 | 0.32 |
| motif 4 | | | | | | | | |
| PPR | 369, R | 372, K | 399, Y | *K* | 0.17 | 0.32 | 0.23 | 0.29 |
| motif 5 | | | | | | | | |
| PPR | 401, E | 404, L | 434, S | *LS | 0.22 | 0.37 | 0.06 | 0.34 |
| motif 6 | | | | | | | | |
| PPR | 436, S | 439, S | 469, E | *SE | 0.58 | 0.07 | 0.10 | 0.25 |
| motif 7 | | | | | | | | |
| PPR | 471, T | 474, D | 505, M | *D* | 0.25 | 0.24 | 0.23 | 0.29 |
| motif 8 | | | | | | | | |
| PPR | 507, N | 510, M | 540, R | *M* | 0.13 | 0.14 | 0.22 | 0.51 |
| motif 9 | | | | | | | | |

The GUN1 protein of Arabidopsis thaliana (SEQ ID NO: 2) has 11 PPR motifs, and the positions of the residues of No. 1 A.A., No. 4 A.A., and No. “ii” (-2) A.A. in the amino acid sequence are as summarized in the following table and FIG. 3.

TABLE 2

| | | | | Code | Base to be | | | |
|---|---|---|---|---|---|---|---|---|
| | | | | (1, 4, | bound (ratio) | | | |
| | A₁ | A₄ | Lᵢᵢ | ii) | A | C | G | T |
| PPR | 234, K | 237, S | 267, 7 | *S* | 0.41 | 0.12 | 0.22 | 0.25 |
| motif 1 | | | | | | | | |
| PPR | 269,Y | 272, S | 302, N | *SN | 0.62 | 0.07 | 0.04 | 0.26 |
| motif 2 | | | | | | | | |
| PPR | 304, V | 307, N | 338, D | VND | 0.06 | 0.21 | 0.24 | 0.31 |
| motif 3 | | | | | | | | |
| PPR | 340, I | 343, N | 373, D | IND | 0.14 | 0.24 | 0.12 | 0.50 |
| motif 4 | | | | | | | | |
| PPR | 375, F | 378, N | 408, N | FNN | 0.24 | 0.21 | 0.24 | 0.31 |
| motif 5 | | | | | | | | |
| PPR | 410, V | 413, S | 443, D | VSD | 0.33 | 0.24 | 0.23 | 0.20 |
| motif 6 | | | | | | | | |
| PPR | 445, V | 448, N | 478, D | VND | 0.06 | 0.21 | 0.06 | 0.66 |
| motif 7 | | | | | | | | |
| PPR | 480, V | 483, N | 513, N | VNN | 0.17 | 0.48 | 0.09 | 0.26 |
| motif 8 | | | | | | | | |
| PPR | 515, L | 518, S | 548, D | *SD | 0.20 | 0.17 | 0.39 | 0.24 |
| motif 9 | | | | | | | | |
| PPR | 550, V | 553, S | 583, N | VSN | 0.57 | 0.09 | 0.05 | 0.30 |
| motif 10 | | | | | | | | |
| PPR | 585, V | 588, N | 620, A | *N* | 0.10 | 0.33 | 0.10 | 0.48 |
| motif 11 | | | | | | | | |

The pTac2 protein of Arabidopsis thaliana (SEQ ID NO: 3) has 15 PPR motifs, and the positions of the residues of No. 1 A.A., No. 4 A.A., and No. “ii” (-2) A.A. in the amino acid sequence are as summarized in the following table and FIG. 3.

TABLE 3

| | | | | Code | Base to be bound | | | |
|---|---|---|---|---|---|---|---|---|
| | A₁ | A₄ | Lᵢᵢ | (1, 4, ii) | A | C | G | T |
| PPR | 106, N | 109, A | 140, N | *AN | 0.45 | 0.18 | 0.05 | 0.32 |
| motif 1 | | | | | | | | |
| PPR | 142, H | 145, T | 175, S | *TS | 0.37 | 0.29 | 0.15 | 0.19 |
| motif 2 | | | | | | | | |
| PPR | 177, F | 180, T | 210, S | *TS | 0.37 | 0.29 | 0.15 | 0.19 |
| motif 3 | | | | | | | | |
| PPR | 212, L | 215, N | 246, D | LND | 0.08 | 0.15 | 0.23 | 0.54 |
| motif 4 | | | | | | | | |
| PPR | 248, V | 251, N | 281, D | VND | 0.06 | 0.21 | 0.06 | 0.66 |
| motif 5 | | | | | | | | |
| PPR | 283, T | 286, S | 316, D | TSD | 0.14 | 0.18 | 0.14 | 0.54 |
| motif 6 | | | | | | | | |
| PPR | 318, T | 321, N | 351, N | TNN | 0.08 | 0.49 | 0.17 | 0.26 |
| motif 7 | | | | | | | | |
| PPR | 353, N | 356, S | 386, D | *SD | 0.20 | 0.17 | 0.39 | 0.24 |
| motif 8 | | | | | | | | |
| PPR | 388, A | 491, N | 421, D | AND | 0.07 | 0.05 | 0.14 | 0.74 |
| motif 9 | | | | | | | | |
| PPR | 423, E | 426, E | 456, S | B.G. | 0.25 | 0.21 | 0.18 | 0.36 |
| motif 10 | | | | | | | | |
| PPR | 458, K | 461, T | 491, S | *TS | 0.37 | 0.29 | 0.15 | 0.19 |
| motif 11 | | | | | | | | |
| PPR | 493, E | 496, H | 526, N | *H* | 0.17 | 0.34 | 0.06 | 0.43 |
| motif 12 | | | | | | | | |
| PPR | 528, D | 531, N | 561, D | *ND | 0.11 | 0.17 | 0.10 | 0.62 |
| motif 13 | | | | | | | | |
| PPR | 563, R | 566, E | 596, S | B.G. | 0.25 | 0.21 | 0.18 | 0.36 |
| motif 14 | | | | | | | | |
| PPR | 598, M | 601, C | 631, I | *C* | 0.55 | 0.10 | 0.21 | 0.14 |
| motif 15 | | | | | | | | |

The DG1 protein of Arabidopsis thaliana (SEQ ID NO: 4) has 10 PPR motifs, and the positions of the residues of No. 1 A.A., No. 4 A.A., and No. “ii” (-2) A.A. in the amino acid sequence are as summarized in the following table and FIG. 3.

TABLE 4

| | | | | Code | Base to be bound | | | |
|---|---|---|---|---|---|---|---|---|
| | A₁ | A₄ | Lᵢᵢ | (1, 4, ii) | A | C | G | T |
| PPR | 256, F | 259, T | 290, D | *TD | 0.10 | 0.10 | 0.67 | 0.13 |
| motif 1 | | | | | | | | |
| PPR | 292, A | 295, H | 340, D | *H* | 0.17 | 0.34 | 0.06 | 0.43 |
| motif 2 | | | | | | | | |
| PPR | 342, V | 345, N | 375, N | VNN | 0.17 | 0.48 | 0.09 | 0.26 |
| motif 3 | | | | | | | | |
| PPR | 377, A | 380, G | 410, K | *G* | 0.29 | 0.13 | 0.31 | 0.27 |
| motif 4 | | | | | | | | |
| PPR | 412, I | 415, K | 445, T | *K* | 0.17 | 0.32 | 0.23 | 0.29 |
| motif 5 | | | | | | | | |
| PPR | 447, S | 450, Y | 481, L | B.G. | 0.25 | 0.21 | 0.18 | 0.36 |
| motif 6 | | | | | | | | |
| PPR | 483, I | 486, T | 515, N | ITN | 0.79 | 0.06 | 0.05 | 0.10 |
| motif 7 | | | | | | | | |
| PPR | 517, G | 520, N | 553, N | *NN | 0.12 | 0.44 | 0.13 | 0.30 |
| motif 8 | | | | | | | | |
| PPR | 555, Y | 558, S | 588, D | YSD | 0.25 | 0.15 | 0.39 | 0.20 |
| motif 9 | | | | | | | | |
| PPR | 590, T | 593, A | 623, H | *AH | 0.41 | 0.08 | 0.07 | 0.45 |
| motif 10 | | | | | | | | |

The GRP23 protein of Arabidopsis thaliana (SEQ ID NO: 5) has 11 PPR motifs, and the positions of the residues of No. 1 A.A., No. 4 A.A., and No. “ii” (-2) A.A. in the amino acid sequence are as summarized in the following table and FIG. 3.

TABLE 5

| | | | | Code | Base to be bound | | | |
|---|---|---|---|---|---|---|---|---|
| | A₁ | A₄ | Lᵢᵢ | (1, 4, ii) | A | C | G | T |
| PPR | 181, F | 184, N | 215, N | FNN | 0.24 | 0.21 | 0.24 | 0.31 |
| motif 1 | | | | | | | | |
| PPR | 217, V | 220, N | 251, S | VNS | 0.07 | 0.61 | 0.05 | 0.27 |
| motif 2 | | | | | | | | |
| PPR | 253, V | 256, R | 286, D | *RD | 0.25 | 0.07 | 0.06 | 0.62 |
| motif 3 | | | | | | | | |
| PPR | 288, T | 291, N | 321, D | TND | 0.14 | 0.08 | 0.07 | 0.71 |
| motif 4 | | | | | | | | |
| PPR | 323, I | 326, A | 356, H | *AH | 0.41 | 0.08 | 0.07 | 0.45 |
| motif 5 | | | | | | | | |
| PPR | 358, P | 361, N | 396, N | *NN | 0.12 | 0.44 | 0.13 | 0.30 |
| motif 6 | | | | | | | | |
| PPR | 398, D | 401, G | 435, D | *GD | 0.09 | 0.09 | 0.59 | 0.25 |
| motif 7 | | | | | | | | |
| PPR | 437, L | 440, C | 470, D | *CD | 0.30 | 0.15 | 0.35 | 0.20 |
| motif 8 | | | | | | | | |
| PPR | 472, P | 475, R | 505, V | *R* | 0.25 | 0.07 | 0.06 | 0.62 |
| motif 9 | | | | | | | | |
| PPR | 507, D | 510, A | 540, D | *AD | 0.10 | 0.22 | 0.39 | 0.29 |
| motif 10 | | | | | | | | |
| PPR | 542, S | 545, D | 575, T | *D* | 0.25 | 0.24 | 0.23 | 0.29 |
| motif 11 | | | | | | | | |

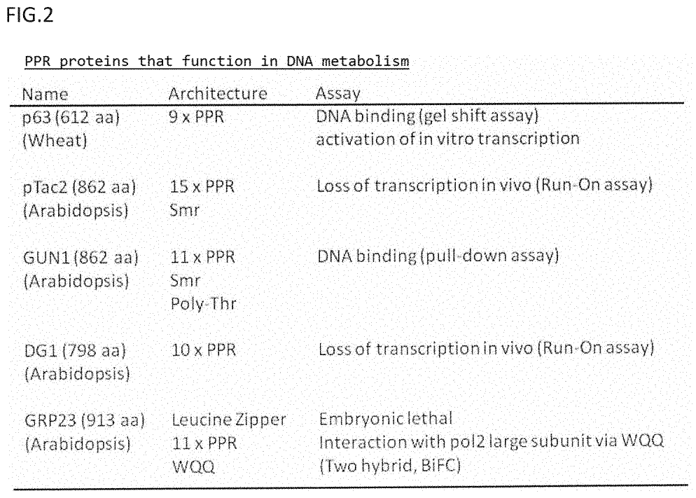

The amino acid frequencies for these positions were confirmed for each protein, and compared with the amino acid frequencies for the same positions of the RNA-binding type motifs. The results are shown in FIG. 2. It became clear that the tendencies of the amino acid frequencies in the PPR motifs of the PPR proteins for which DNA-binding property is suggested, and the RNA-binding type motifs substantially agreed with each other. That is, it became clear that the PPR proteins that act to bind to DNA bind with nucleic acids according to same sequence rules as those of the PPR proteins that act to bind to RNA, and the RNA recognition codes described in the pending patent application of the inventors of the present invention (PCT/JP2012/077274) can be applied as the DNA recognition codes of the PPR proteins that act to bind to DNA.

With reference to the RNA recognition codes described in the non-patent document (Yagi, Y. et al., Plos One, 2013, 8, e57286), the DNA-binding type PPR motifs that selectively bind to each corresponding base were evaluated. More precisely, a chi square test was performed on the basis of occurrence nucleotide frequencies shown in Table 6 and expected nucleotide frequencies calculated from the background frequencies. The test was performed for each base (NT), purine or pyrimidine (AG or CT, PY), hydrogen bond group (AT or GC, HB), or amino or keto form (AC or GT). Significant value was defined as P<0.06 (5E-02, 5% significance level), and when a significant value was obtained in any of the tests, the combination of No. 1 amino acid, No. 4 amino acid, and No. “ii” (-2) amino acid was chosen.

TABLE 6
Base selectivity of DNA-binding code

| NSRs | occurrence | Probabilitiy matrix | | | | subtraction for background | | | |
|---|---|---|---|---|---|---|---|---|---|
| (1, 4, ii) | of the NSR(s) | A | C | G | T | A | C | G | T |
| *GD | 14 | 0.10 | 0.06 | 0.57 | 0.28 | −0.16 | −0.15 | 0.40 | −0.08 |
| EGD | 8 | 0.07 | 0.05 | 0.69 | 0.19 | −1.19 | −1.16 | 0.52 | −0.17 |
| *GN | 11 | 0.55 | 0.10 | 0.04 | 0.31 | 0.29 | −0.11 | −0.13 | −0.05 |
| EGN | 5 | 0.63 | 0.06 | 0.05 | 0.25 | 0.37 | −0.15 | −0.12 | −0.11 |
| *GS | 3 | 0.57 | 0.23 | 0.06 | 0.14 | 0.31 | 0.02 | −0.11 | −0.22 |
| *I* | 15 | 0.15 | 0.29 | 0.10 | 0.45 | −0.11 | 0.08 | −0.07 | 0.09 |
| *IN | 4 | 0.17 | 0.28 | 0.06 | 0.50 | −0.09 | 0.07 | −0.11 | 0.14 |
| *L* | 23 | 0.20 | 0.30 | 0.03 | 0.47 | −0.06 | 0.09 | −0.14 | 0.11 |
| *LD | 6 | 0.19 | 0.47 | 0.05 | 0.28 | −0.07 | 0.26 | −0.12 | −0.08 |
| *LK | 3 | 0.09 | 0.08 | 0.06 | 0.77 | −0.17 | −0.13 | −0.11 | 0.41 |
| *M* | 10 | 0.14 | 0.15 | 0.16 | 0.56 | −0.12 | −0.06 | −0.02 | 0.20 |
| *MD | 9 | 0.15 | 0.13 | 0.17 | 0.55 | −0.11 | −0.08 | 0.00 | 0.19 |
| IMD | 4 | 0.09 | 0.24 | 0.06 | 0.62 | −0.17 | 0.03 | −0.11 | 0.26 |
| *N* | 147 | 0.11 | 0.33 | 0.10 | 0.45 | −0.15 | 0.12 | −0.07 | 0.09 |
| ND | 72 | 0.11 | 0.18 | 0.10 | 0.61 | −0.15 | −0.03 | −0.07 | 0.25 |
| FND | 13 | 0.23 | 0.19 | 0.10 | 0.49 | −0.03 | −0.02 | −0.07 | 0.13 |
| GND | 3 | 0.09 | 0.08 | 0.06 | 0.77 | −0.17 | −0.13 | −0.11 | 0.41 |
| IND | 5 | 0.22 | 0.13 | 0.06 | 0.60 | −0.04 | −0.08 | −0.12 | 0.24 |
| TND | 3 | 0.15 | 0.08 | 0.06 | 0.72 | −0.11 | −0.13 | −0.11 | 0.36 |
| VND | 23 | 0.06 | 0.25 | 0.06 | 0.63 | −0.20 | 0.04 | −0.11 | 0.27 |
| YND | 6 | 0.08 | 0.30 | 0.11 | 0.52 | −0.18 | 0.09 | −0.06 | 0.16 |
| *NN | 34 | 0.15 | 0.45 | 0.14 | 0.27 | −0.11 | 0.24 | −0.03 | −0.09 |
| INN | 7 | 0.12 | 0.49 | 0.05 | 0.34 | −0.14 | 0.28 | −0.12 | −0.02 |
| SNN | 3 | 0.09 | 0.60 | 0.06 | 0.24 | −0.17 | 0.39 | −0.11 | −0.12 |
| VNN | 10 | 0.20 | 0.53 | 0.04 | 0.23 | −0.06 | 0.32 | −0.13 | −0.13 |
| *NS | 13 | 0.11 | 0.47 | 0.07 | 0.36 | −0.15 | 0.26 | −0.10 | 0.00 |
| VNS | 5 | 0.08 | 0.66 | 0.05 | 0.21 | −0.18 | 0.45 | −0.12 | −0.15 |
| *NT | 13 | 0.12 | 0.52 | 0.13 | 0.24 | −0.14 | 0.31 | −0.04 | −0.12 |
| VNT | 5 | 0.08 | 0.57 | 0.05 | 0.30 | −0.18 | 0.36 | −0.12 | −0.06 |
| *NW | 11 | 0.14 | 0.32 | 0.13 | 0.41 | −0.12 | 0.11 | −0.04 | 0.05 |
| INW | 3 | 0.09 | 0.29 | 0.06 | 0.56 | −0.17 | 0.08 | −0.11 | 0.20 |
| *P* | 17 | 0.10 | 0.06 | 0.11 | 0.73 | −0.16 | −0.15 | −0.06 | 0.37 |
| *PD | 9 | 0.06 | 0.09 | 0.10 | 0.75 | −0.20 | −0.12 | −0.07 | 0.39 |
| FPD | 3 | 0.09 | 0.08 | 0.06 | 0.77 | −0.17 | −0.13 | −0.11 | 0.41 |
| YPD | 3 | 0.09 | 0.08 | 0.06 | 0.77 | −0.17 | −0.13 | −0.11 | 0.41 |
| *S* | 49 | 0.38 | 0.13 | 0.20 | 0.29 | 0.12 | −0.08 | 0.03 | −0.07 |
| *SN | 18 | 0.63 | 0.08 | 0.05 | 0.24 | 0.37 | −0.13 | −0.12 | −0.12 |
| FSN | 7 | 0.63 | 0.13 | 0.08 | 0.16 | 0.37 | −0.08 | −0.09 | −0.20 |
| VSN | 6 | 0.60 | 0.10 | 0.05 | 0.25 | 0.34 | −0.11 | −0.12 | −0.11 |
| *T* | 86 | 0.45 | 0.09 | 0.31 | 0.15 | 0.19 | −0.12 | 0.14 | −0.21 |
| *TD | 32 | 0.13 | 0.12 | 0.61 | 0.14 | −0.13 | −0.09 | 0.44 | −0.22 |
| VTD | 7 | 0.07 | 0.06 | 0.67 | 0.20 | −0.19 | −0.15 | 0.50 | −0.16 |
| *TN | 31 | 0.66 | 0.08 | 0.13 | 0.13 | 0.40 | −0.13 | −0.04 | −0.23 |
| FTN | 4 | 0.75 | 0.07 | 0.06 | 0.12 | 0.49 | −0.14 | −0.11 | −0.24 |
| ITN | 5 | 0.77 | 0.06 | 0.05 | 0.11 | 0.51 | −0.15 | −0.12 | −0.25 |
| VTN | 10 | 0.63 | 0.13 | 0.15 | 0.09 | 0.37 | −0.08 | −0.02 | −0.27 |
| *V* | 48 | 0.29 | 0.21 | 0.08 | 0.43 | 0.03 | 0.00 | −0.09 | 0.07 |
| IVD | 3 | 0.31 | 0.50 | 0.06 | 0.14 | 0.05 | 0.29 | −0.11 | −0.22 |
| VG | 5 | 0.22 | 0.48 | 0.05 | 0.25 | −0.04 | 0.27 | −0.12 | −0.11 |
| *VT | 4 | 0.25 | 0.07 | 0.06 | 0.62 | −0.01 | −0.14 | −0.11 | 0.26 |

| | | | | |
|---|---|---|---|---|
| Background frequency | 0.26 | 0.21 | 0.17 | 0.36 |

In Table 1, the combinations of the amino acids that showed significant base selectivity were mentioned. That is, these results mean that the PPR motifs having the amino acid species of the No. 1 amino acid, No. 4 amino acid, and No. “ii” (-2) amino acid (“NSRs (1, 4, and ii)” in the table) that provided a significant P value are PPR motifs that impart base-selective binding ability, and a larger “positive” value obtained after the subtraction of the background means higher base selectivity for the base. Among the No. 1 amino acid, No. 4 amino acid, and No. “ii” (-2) amino acid, the No. 4 amino acid most strongly affects the base selectivity, the No. “ii” (-2) amino acid affects the base selectivity next strongly, and the No. 1 amino acid most weakly affects the base selectivity among the three amino acids.

### Example 2: Evaluation of Sequence-Specific DNA-Binding Ability PPR Molecules

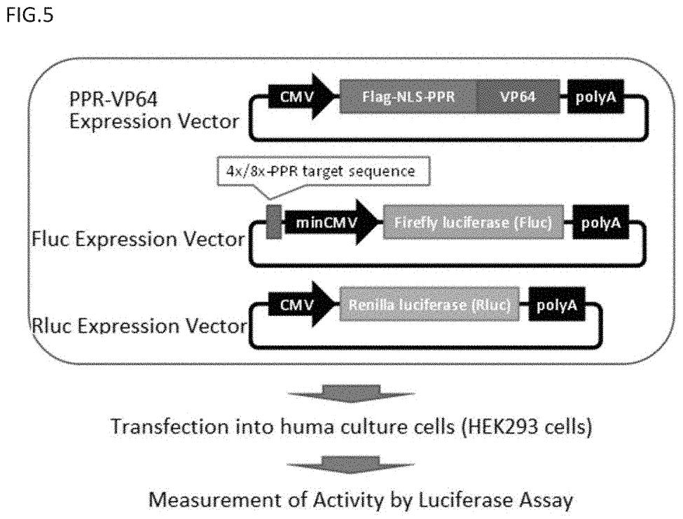

In this example, artificial transcription factors were prepared by fusing VP64, which is a transcription activation domain, to the three kinds of DNA-binding type (expectedly) PPR molecules, p63, pTac2, and GUN1, and by examining whether they could activate luciferase reporters each having a corresponding target sequence in a human cultured cell, whether the PPR molecules had a sequence-specific DNA-binding ability or not was determined (FIG. 5).

(Experimental Method)

1. Preparation of PPR-VP64 Expression Vector

Only the parts corresponding to the PPR motifs in the coding sequences of p63, pTac2, and GUN1 were prepared by artificial synthesis. For the DNA synthesis, the artificial gene synthesis service of Biomatik was used. The pCS2P vector having the CMV promoter was used as a backbone vector, and each synthesized PPR sequence was inserted into it. Further, the Flag tag and nuclear transfer signal were inserted at the N-terminus of the PPR sequence, and the VP64 sequence was inserted at the C-terminus of the same. The produced sequences of p63-VP64, pTac2-VP64, and GUN1-VP64 are shown in Sequence Listing as SEQ ID NOS: 7 to 9.

2. Preparation of Reporter Vector Having PPR Target Sequence

A reporter vector (pminCMV-luc2, SEQ ID NO: 10) was prepared, in which the firefly luciferase gene was ligated downstream from the Minimal CMV promoter, and a multi-cloning site was placed upstream of the promoter. The predicted target sequence of each PPR was inserted into the vector at the multi-cloning site. The target sequence of each PPR (TCTATCACT for p63, AACTTTCGTCACTCA for pTac2, and AATTTGTCGAT for GUN1, SEQ ID NOS: 11 to 13 in Sequence Listing) was determined by predicting the motif-DNA recognition codes of DNA-binding type PPR from the motif-RNA recognition codes observed in the RNA-binding type PPR. For each PPR, sequences containing 4 or 8 of target sequences were prepared, and used in the following assay. The nucleotide sequences of the vectors are shown as SEQ ID NOS: 14 to 19 in Sequence Listing.

3. Transfection into HEK293 T Cell

The PPR-VP64 expression vector prepared in the section 1, the firefly luciferase expression vector prepared in the section 2, and the pRL-CMV vector (expression vector for Renilla luciferase, Promega) as a reference were introduced by using Lipofectamine LTX (Life Technologies). The DMEM medium (25 μl) was added to each well of a 96-well plate, and a mixture containing the PPR-VP64 expression vector (400 ng), firefly luciferase expression vector (100 ng), and pRL-CMV vector (20 ng) was further added. Then, a mixture of the DMEM medium (25 μl) and Lipofectamine LTX (0.7 μl) was added to each well, the plate was left standing at room temperature for 30 minutes, then 6×10⁴ of the HEK293 T cells suspended in the DMEM medium containing 15% fetal bovine serum (100 μl) were added, and the cells were cultured at 37° C. in a CO₂ incubator for 24 hours.

4. Luciferase Assay

Luciferase assay was performed by using Dual-Glo Luciferase Assay System (Promega) in accordance with the instructions attached to the kit. For the measurement of the luciferase activity, Tri Star LB 941 Plate Reader (Berthold) was used.

(Results and Discussion)

The luciferase activity was compared for the cases of introducing pTac2-VP64 or GUN1-VP64 together with pminCMV-luc2 for a negative control, or the reporter vector having 4 or 8 target sequences (table mentioned below, FIG. 6). The comparison of the activity was performed on the basis of standardized scores obtained by dividing the measured values obtained with Fluc (firefly luciferase) with the measured value obtained with Rluc (Renilla luciferase) as the reference (Fluc/Rluc). As a result, there was observed a tendency that the activity increased with increase of the number of the target sequence for the both cases, and thus it was verified that each of the PPR-VP64 molecules specifically bound to each target sequence, and functioned as a site-specific transcription activator.

| | Fluc reporter | PPR-VP64 | Reference | Fluc | Rluc | Fluc/Rluc | Fold activation |
|---|---|---|---|---|---|---|---|
| pTac2-VP64 | pminCMV-luc2 | pTac2-VP64 | pRL-CMV | 47744 | 4948 | 9.649151172 | 1 |
| (negative control) | | | | | | | |
| pTac2-VP64 | pTac2-4x target | pTac2-VP64 | pRL-CMV | 133465 | 4757 | 28.05654824 | 2.907670089 |
| (4x target) | | | | | | | |
| pTac2-VP64 | pTac2-8x target | pTac2-VP64 | pRL-CMV | 189146 | 4011 | 47.15681875 | 4.887146849 |
| (8x target) | | | | | | | |
| GUN1-VP64 | pminCMV-luc2 | GUN1-VP64 | pRL-CMV | 29590 | 3799 | 7.788891814 | 1 |
| (negative control) | | | | | | | |
| GUN1-VP64 | GUN1-4x target | GUN1-VP64 | pRL-CMV | 61070 | 2727 | 22.39457279 | 2.875193715 |
| (4x target) | | | | | | | |
| GUN1-VP64 | GUN1-8x target | GUN1-VP64 | pRL-CMV | 66982 | 2731 | 24.52654705 | 3.14891356 |
| (8x target) | | | | | | | |

What is claimed is:

1. A method for designing a DNA-binding protein that can bind in a DNA base-selective manner or a DNA base sequence-specific manner, the method comprising:
determining an amino acid sequence of the DNA-binding protein,
wherein the DNA-binding protein contains one or more motifs having a structure of the following formula 1:
(Helix A)-X-(Helix B)-L (Formula 1)
(wherein, in the formula 1:
Helix A is a part that can form an α-helix structure;
X does not exist, or is a part consisting of 1 to 9 amino acids;
Helix B is a part that can form an α-helix structure; and
L is a part consisting of 2 to 7 amino acids),
wherein,
under the following definitions:
the first amino acid of Helix A is referred to as Number 1 amino acid (Number 1 AA), the fourth amino acid as Number 4 amino acid (Number 4 AA), and
when a next PPR motif (Mₙ₊₁) contiguously exists on the C-terminus side of the PPR motif (Mₙ) (when there is no amino acid insertion between the PPR motifs), the −2nd amino acid counted from the end (C-terminus side) of the amino acids constituting the PPR motif (Mₙ);
when a non-PPR motif consisting of 1 to 20 amino acids exists between the PPR motif (Mₙ) and the next PPR motif (Mₙ₊₁) on the C-terminus side, the amino acid locating upstream of the first amino acid of the next PPR motif (Mₙ₊₁) by 2 positions, i.e., the −2nd amino acid; or
when any next PPR motif (Mₙ₊₁) does not exist on the C-terminus side of the PPR motif (Mₙ), or 21 or more amino acids constituting a non-PPR motif exist between the PPR motif (Mₙ) and the next PPR motif (Mₙ₊₁) on the C-terminus side, the 2nd amino acid counted from the end (C-terminus side) of the amino acids constituting the PPR motif (Mₙ)
is referred to as Number “ii” (-2) amino acid (Number “ii” (-2) AA),
each PPR motif (Mₙ) contained in the protein is a PPR motif having a specific combination of amino acids as the three amino acids of Number 1 AA, Number 4 AA, and Number “ii” (-2) AA,
the combination of the three amino acids of Number 1 AA, Number 4 AA, and Number “ii” (-2) AA in each motif is a combination corresponding to a target DNA base of the target DNA base sequence, and the combination of amino acids is determined according to any one of the following definitions:
(2-1) when the target DNA base to which the PPR motif binds is the three amino acids, Number 1 AA, Number 4 AA, and Number “ii” (-2) AA are an arbitrary amino acid, glycine, and aspartic acid, respectively;
(2-2) when the target DNA base to which the PPR motif binds is the three amino acids, Number 1 AA, Number 4 AA, and Number “ii” (-2) AA, are glutamic acid, glycine, and aspartic acid, respectively;
(2-3) when the target DNA base to which the PPR motif binds is A, the three amino acids, Number 1 AA, Number 4 AA, and Number “ii” (-2) AA, are an arbitrary amino acid, glycine, and asparagine, respectively;
(2-4) when the target DNA base to which the PPR motif binds is A, the three amino acids, Number 1 AA, Number 4 AA, and Number “ii” (-2) AA, are glutamic acid, glycine, and asparagine, respectively;
(2-5) when the target DNA base to which the PPR motif binds is A, the three amino acids, Number 1 AA, Number 4 AA, and Number “ii” (-2) AA, are an arbitrary amino acid, glycine, and serine, respectively;
(2-6) when the target DNA base to which the PPR motif binds is T or C, the three amino acids, Number 1 AA, Number 4 AA, and Number “ii” (-2) AA, are an arbitrary amino acid, isoleucine, and an arbitrary amino acid, respectively;
(2-7) when the target DNA base to which the PPR motif binds is T, the three amino acids, Number 1 AA, Number 4 AA, and Number “ii” (-2) AA, are an arbitrary amino acid, isoleucine, and asparagine, respectively;
(2-8) when the target DNA base to which the PPR motif binds is T or C, the three amino acids, Number 1 AA, Number 4 AA, and Number “ii” (-2) AA, are an arbitrary amino acid, leucine, and an arbitrary amino acid, respectively;
(2-9) when the target DNA base to which the PPR motif binds is C, the three amino acids, Number 1 AA, Number 4 AA, and Number “ii” (-2) AA, are an arbitrary amino acid, leucine, and aspartic acid, respectively; (2-10) when the target DNA base to which the PPR motif binds is T, the three amino acids, Number 1 AA, Number 4 AA, and Number “ii” (-2) AA, are an arbitrary amino acid, leucine, and lysine, respectively;
(2-11) when the target DNA base to which the PPR motif binds is the target DNA base to which the PPR motif binds is T, the three amino acids, Number 1 AA, Number 4 AA, and Number “ii” (-2) AA, are an arbitrary amino acid, methionine, and an arbitrary amino acid, respectively;
(2-12) when the target DNA base to which the PPR motif binds is T, the three amino acids, Number 1 AA, Number 4 AA, and Number “ii” (-2) AA, are an arbitrary amino acid, methionine, and aspartic acid, respectively;
(2-13) when the target DNA base to which the PPR motif binds is C, the three amino acids, Number 1 AA, Number 4 AA, and Number “ii” (-2) AA, are isoleucine, methionine, and aspartic acid, respectively;
(2-14) when the target DNA base to which the PPR motif binds is C or T, the three amino acids, Number 1 AA, Number 4 AA, and Number “ii” (-2) AA, are an arbitrary amino acid, asparagine, and an arbitrary amino acid, respectively;
(2-15) when the target DNA base to which the PPR motif binds is T, the three amino acids, Number 1 AA, Number 4 AA, and Number “ii” (-2) AA, are an arbitrary amino acid, asparagine, and aspartic acid, respectively;
(2-16) when the target DNA base to which the PPR motif binds is T, the three amino acids, Number 1 AA, Number 4 AA, and Number “ii” (-2) AA, are phenylalanine, asparagine, and aspartic acid, respectively;
(2-17) when the target DNA base to which the PPR motif binds is T, the three amino acids, Number 1 AA, Number 4 AA, and Number “ii” (-2) AA, are glycine, asparagine, and aspartic acid, respectively;
(2-18) when the target DNA base to which the PPR motif binds is T, the three amino acids, Number 1 AA, Number 4 AA, and Number “ii” (-2) AA, are isoleucine, asparagine, and aspartic acid, respectively;
(2-19) when the target DNA base to which the PPR motif binds is T, the three amino acids, Number 1 AA, Number 4 AA, and Number “ii” (-2) AA, are threonine, asparagine, and aspartic acid, respectively;
(2-20) when the target DNA base to which the PPR motif binds is T, the three amino acids, Number 1 AA, Number 4 AA, and Number “ii” (-2) AA are valine, asparagine, and aspartic acid, respectively;
(2-21) when the target DNA base to which the PPR motif binds is T, the three amino acids, Number 1 AA, Number 4 AA, and Number “ii” (-2) AA are tyrosine, asparagine, and aspartic acid, respectively;
(2-22) when the target DNA base to which the PPR motif binds is C, the three amino acids, Number 1 AA, Number 4 AA, and Number “ii” (-2) AA, are an arbitrary amino acid, asparagine, and asparagine, respectively;
(2-23) when the target DNA base to which the PPR motif binds is C, the three amino acids, Number 1 AA, Number 4 AA, and Number “ii” (-2) AA, are isoleucine, asparagine, and asparagine, respectively;
(2-24) when the target DNA base to which the PPR motif binds is C, the three amino acids, Number 1 AA, Number 4 AA, and Number “ii” (-2) AA, are serine, asparagine, and asparagine, respectively;
(2-25) when the target DNA base to which the PPR motif binds is C, the three amino acids, Number 1 AA, Number 4 AA, and Number “ii” (-2) AA, are valine, asparagine, and asparagine, respectively;
(2-26) when the target DNA base to which the PPR motif binds is C, the three amino acids, Number 1 AA, Number 4 AA, and Number “ii” (-2) AA, are an arbitrary amino acid, asparagine, and serine, respectively;
(2-27) when the target DNA base to which the PPR motif binds is C, the three amino acids, Number 1 AA, Number 4 AA, and Number “ii” (-2) AA, are valine, asparagine, and serine, respectively;
(2-28) when the target DNA base to which the PPR motif binds is C, the three amino acids, Number 1 AA, Number 4 AA, and Number “ii” (-2) AA, are an arbitrary amino acid, asparagine, and threonine, respectively;
(2-29) when the target DNA base to which the PPR motif binds is C, the three amino acids, Number 1 AA, Number 4 AA, and Number “ii” (-2) AA, are valine, asparagine, and threonine, respectively;
(2-30) when the target DNA base to which the PPR motif binds is C, the three amino acids, Number 1 AA, Number 4 AA, and Number “ii” (-2) AA, are an arbitrary amino acid, asparagine, and tryptophan, respectively;
(2-31) when the target DNA base to which the PPR motif binds is T, the three amino acids, Number 1 AA, Number 4 AA, and Number “ii” (-2) AA, are isoleucine, asparagine, and tryptophan, respectively;
(2-32) when the target DNA base to which the PPR motif binds is T, the three amino acids, Number 1 AA, Number 4 AA, and Number “ii” (-2) AA, are an arbitrary amino acid, proline, and an arbitrary amino acid, respectively;
(2-33) when the target DNA base to which the PPR motif binds is T, the three amino acids, Number 1 AA, Number 4 AA, and Number “ii” (-2) AA, are an arbitrary amino acid, proline, and aspartic acid, respectively;
(2-34) when the target DNA base to which the PPR motif binds is T, the three amino acids, Number 1 AA, Number 4 AA, and Number “ii” (-2) AA, are phenylalanine, proline, and aspartic acid, respectively;
(2-35) when the target DNA base to which the PPR motif binds is T, the three amino acids, Number 1 AA, Number 4 AA, and Number “ii” (-2) AA, are tyrosine, proline, and aspartic acid, respectively;
(2-36) when the target DNA base to which the PPR motif binds is A or the three amino acids, Number 1 AA, Number 4 AA, and Number “ii” (-2) AA, are an arbitrary amino acid, serine, and an arbitrary amino acid, respectively;
(2-37) when the target DNA base to which the PPR motif binds is A, the three amino acids, Number 1 AA, Number 4 AA, and Number “ii” (-2) AA, are an arbitrary amino acid, serine, and asparagine, respectively;
(2-38) when the target DNA base to which the PPR motif binds is A, the three amino acids, Number 1 AA, Number 4 AA, and Number “ii” (-2) AA, are phenylalanine, serine, and asparagine, respectively;
(2-39) when the target DNA base to which the PPR motif binds is A, the three amino acids, Number 1 AA, Number 4 AA, and Number “ii” (-2) AA, are valine, serine, and asparagine, respectively;
(2-40) when the target DNA base to which the PPR motif binds is A or the three amino acids, Number 1 AA, Number 4 AA, and Number “ii” (-2) AA, are an arbitrary amino acid, threonine, and an arbitrary amino acid, respectively;
(2-41) when the target DNA base to which the PPR motif binds is the three amino acids, Number 1 AA, Number 4 AA, and Number “ii” (-2) AA, are an arbitrary amino acid, threonine, and aspartic acid, respectively;
(2-42) when the target DNA base to which the PPR motif binds is the three amino acids, Number 1 AA, Number 4 AA, and Number “ii” (-2) AA, are valine, threonine, and aspartic acid, respectively;
(2-43) when the target DNA base to which the PPR motif binds is A, the three amino acids, Number 1 AA, Number 4 AA, and Number “ii” (-2) AA, are an arbitrary amino acid, threonine, and asparagine, respectively;
(2-44) when the target DNA base to which the PPR motif binds is A, the three amino acids, Number 1 AA, Number 4 AA, and Number “ii” (-2) AA, are phenylalanine, threonine, and asparagine, respectively;
(2-45) when the target DNA base to which the PPR motif binds is A, the three amino acids, Number 1 AA, Number 4 AA, and Number “ii” (-2) AA, are isoleucine, threonine, and asparagine, respectively;
(2-46) when the target DNA base to which the PPR motif binds is A, the three amino acids, Number 1 AA, Number 4 AA, and Number “ii” (-2) AA, are valine, threonine, and asparagine, respectively;
(2-47) when the target DNA base to which the PPR motif binds is A, C, or T, the three amino acids, Number 1 AA, Number 4 AA, and Number “ii” (-2) AA, are an arbitrary amino acid, valine, and an arbitrary amino acid, respectively;
(2-48) when the target DNA base to which the PPR motif binds is C, the three amino acids, Number 1 AA, Number 4 AA, and Number “ii” (-2) AA, are isoleucine, valine, and aspartic acid, respectively;
(2-49) when the target DNA base to which the PPR motif binds is C, the three amino acids, Number 1 AA, Number 4 AA, and Number “ii” (-2) AA, are an arbitrary amino acid, valine, and glycine, respectively; and
(2-50) when the target DNA base to which the PPR motif binds is T, the three amino acids, Number 1 AA, Number 4 AA, and Number “ii” (-2) AA, are an arbitrary amino acid, valine, and threonine, respectively.

2. The method according to claim 1, wherein the one or more PPR motifs are any group of motifs selected from
9 PPR motifs belonging to the p63 protein consisting of the amino acid sequence of SEQ ID NO: 1,
11 PPR motifs belonging to the GUN1 protein consisting of the amino acid sequence of SEQ ID NO: 2,
15 PPR motifs belonging to the pTac2 protein consisting of the amino acid sequence of SEQ ID NO: 3,
10 PPR motifs belonging to the DG1 protein consisting of the amino acid sequence of SEQ ID NO: 4, and
11 PPR motifs belonging to the GRP23 protein consisting of the amino acid sequence of SEQ ID NO: 5.

3. A method for preparing the DNA-binding protein designed by the method according to claim 1, comprising:
determining a nucleic acid sequence coding for an amino acid sequence of the designed DNA-binding protein,
cloning said nucleic acid sequence, and
preparing a transformant which produces the DNA-binding protein.

4. The method according to claim 3, wherein the one or more PPR motifs are any group of motifs selected from
9 PPR motifs belonging to the p63 protein consisting of the amino acid sequence of SEQ ID NO: 1,
11 PPR motifs belonging to the GUN1 protein consisting of the amino acid sequence of SEQ ID NO: 2,
15 PPR motifs belonging to the pTac2 protein consisting of the amino acid sequence of SEQ ID NO: 3,
10 PPR motifs belonging to the DG1 protein consisting of the amino acid sequence of SEQ ID NO: 4, and
11 PPR motifs belonging to the GRP23 protein consisting of the amino acid sequence of SEQ ID NO: 5.
